(19) United States
(12) Patent Application Publication
(10) Pub. No.: US 2023/0363363 A1
(43) Pub. Date: Nov. 16, 2023

(54) AUTOMATED OR SEMI-AUTOMATED REARING, SORTING AND COUNTING OF PUPAE AND LARVAE

(71) Applicant: Senecio Ltd., Kfar-Saba (IL)

(72) Inventors: Hanan LEPEK, Kfar-Saba (IL); Ido NUSBAUM, Hod Hasharon (IL); Tamir LEV, Haifa (IL)

(73) Assignee: Senecio Ltd., Kfar-Saba (IL)

(21) Appl. No.: 18/029,389

(22) PCT Filed: Sep. 30, 2021
(86) PCT No.: PCT/IL2021/051183

§ 371 Date: Mar. 30, 2023

Related U.S. Application Data
(60) Provisional application No. 63/085,180, filed on Sep. 30, 2020.
(60) Provisional application No. 63/089,079, filed on Oct. 8, 2020.

(51) Int. Cl. A01K 67/033; A01K 61/80

(52) U.S. Cl. CPC A01K 67/033; A01K 61/80; A01K 2227/706

(57) ABSTRACT

Apparatus for rearing of aquatic insects, comprises one or more water trays filled with water and larvae being reared, a water inlet leading to the water surface and a water outlet leading from the water surface, the water inlet closed with an inlet valve and the water outlet closed with an outlet valve. The tray has a mesh, and the insects reared in water in the tray are above the mesh. The tray has a water cleaning configuration in which the inlet and outlet valves are open to flush clean water through the tray while the mesh holds the insects so that they do not get flushed out as the water is changed.

### RELATED APPLICATION/S

This application claims the benefit of priority of U.S. Provisional Patent Application No. 63/085,180 filed on 30 Sep. 2020 and of U.S. Provisional Patent Application No. 63/089,079 filed on 8 Oct. 2020, the contents of which are incorporated herein by reference in their entirety.

### FIELD AND BACKGROUND OF THE INVENTION

The present invention, in some embodiments thereof, relates to constructions, systems and methods for automated or semi-automated rearing sorting and counting of pupae and larvae and, more particularly, but not exclusively, to rearing, sorting and counting of mosquitoes.

Rearing of large numbers of mosquitoes, in the order of millions and more, per week, is essential for large-scale sterile mosquito insertion, in which sterile male mosquitoes are required to be released over large areas. In some projects, both male and female mosquitoes are released in order to support mosquito population replacement.

Common for this application is the need to be able to rear efficiently and in mass with minimum costs and minimum labor efforts, to produce large numbers of mosquito larva, with maximum yield and minimum death of the larva.

Currently, rearing of large numbers of larva is a very manual process, requiring feeding of the larva according to the specific stage of their life cycle. The mosquito larva goes through four instars before it pupates and becomes a pupa—the larva molts (sheds its skin) four times and the stages between molts are called instars. When the 4^{th} instar molts it becomes a pupa. Then from the pupa, the adult mosquito emerges to become a full-grown adult mosquito.

Furthermore, rearing of mosquitoes requires the handling of mosquitoes at their pupa and larva stage. Counting of larvae is required in order to calculate required amounts of food per rearing tray. Counting the number of pupae is required in order to be able to load estimated numbers of pupae into emergence chambers.

Separating the pupa from the larva during the rearing process may also be required in order to prevent loading of larva together with pupae into emergence chambers, when only pupae are supposed to be loaded.

For sterile insect technique programs, which require release of males only, pupae are not only counted but also sex sorted between males and females. All of the above is currently processed manually, which requires a lot of time and effort.

As the pupae and larva may swim within the water container they are stored in, it is difficult to count all the moving objects in the water, and moreover given the fact that pupae and larva may submerge in the water, the three-dimensional aspect only adds to the difficulty of ensuring that all objects are counted.

When an object approaches the water (e.g. a collection cup) pupae and larva tend to swim away to get away from the danger, hence it is difficult to pick up from the water a specific number of counted pupa and/or larva.

Today, pupae and larva separation including pupae sex separation process are considered labor intensive tasks.

### SUMMARY OF THE INVENTION

The present embodiments may provide automation in rearing of aquatic insect larvae and pupae and in sex sorting. Trays or racks of trays are provided with robots which fill the trays with larvae, and provide food at regular intervals. Automated cleaning and emptying sequences are provided that do not harm the insects and detection of the state of the tray is detected so that operations are carried out when appropriate for that tray. Furthermore an automated counting and classification procedure is provided to count and classify the insects without harming them. According to an aspect of some embodiments of the present invention there is provided apparatus for rearing of aquatic insects, comprising at least one water tray, the tray having an interior tray surface and being fillable with water to provide a water surface, a water inlet leading to the water surface and a water outlet leading from the water surface, the water inlet closed with an inlet valve and the water outlet closed with an outlet valve, and mesh, the tray being arranged such that insects reared in water in the tray are above the mesh, and wherein the tray has a water cleaning configuration in which the inlet and outlet valves are open to flush clean water through the tray while the mesh holds the insects.

In embodiments, the mesh is held in a frame around the tray, the frame configured to raise the mesh for the cleaning configuration.

Alternatively, the mesh is held still and the water surface is varied.

In embodiments, the tray comprises at least one rail, and a robot configured to travel a length of the tray along the rail.

In embodiments, the robot comprises a first dispensing container to dispense larvae into the at least one tray and a second dispensing container to dispense food into the tray.

Embodiments may have an emptying mode, wherein the mesh is flush with the inner tray surface and the outlet valve is opened to empty pupae and water from the tray.

Embodiments may have an image sensor and processing software to detect that a threshold level of larvae have pupated and to trigger the emptying mode.

Embodiments may comprise a rack, holding a plurality of the trays, and at least one robot for processing the trays.

In embodiments, the robot comprises a gripper for pulling a selected one of the plurality of trays and pulling the tray to a feeding position extending in a first direction from the rack, the robot further comprising a feeding attachment for dispensing food into the tray.

The robot may have a piston for pushing a selected one of the trays into a pouring position to empty the tray into a collection container.

In embodiments, the rack comprises guide rails for guiding the tray into the pouring position.

Embodiments may comprise an image sensor and image processing for determining levels of pupation in respective ones of the plurality of trays, thereby to empty respective trays only when a threshold level of pupation is met.

In embodiments, the robot comprises a vibrator for vibrating the robot during dispensing to provide even dispensing in the tray.

Embodiments may comprise an odor detector, the odor detector configured to trigger the cleaning configuration upon detection of a threshold level of a predetermined odor.

Embodiments may comprise successive trays, each with progressively larger mesh, each mesh sized for a succeeding instar, and the apparatus being configured to enter an emptying mode wherein the mesh is flush with the inner tray surface and the outlet valve is opened to empty larvae and water from the tray upon detection of a threshold number of larvae entering a next instar stage, the trays being configured that each tray empties into a next one of the successive trays.

According to a second aspect of the present invention there is provided apparatus for counting and/or classification of aquatic insects, comprising:
a drainable imaging area;
a funneling mechanism for funneling water containing the aquatic insects onto the drainable imaging area; and
a sensor at the drainable imaging area for sensing the aquatic insects after the water is drained.

In embodiments, the drainable imaging area comprises a gated entrance for controllable entry of the aquatic insects.

In embodiments, the drainable imaging area comprises a movable surface to move the insects from the gated entrance to the sensor, the movable surface comprising drainage gaps for draining the water whilst retaining the insects.

In embodiments, the drainage gaps are mesh on the movable surface.

In embodiments, the funneling mechanism is a sloped surface with guide walls.

In embodiments, the funneling mechanism is configured to receive the water and aquatic insects from a container tipped onto the funneling mechanism by a tipping mechanism.

Embodiments may carry out sex sorting of pupae by imaging the pupae and classifying the pupae into at least one of two classes of male and female, and placing individuals of one of the classes into a container designated for the respective class.

Classifying may be based on a detected pupa size or on a morphological feature, such as a tapering shape of an abdominal extremity of a pupa.

Alternatively or additionally, sex sorting of larvae may be achieved by engineering the larvae to fluoresce differentially depending on whether they are male or female, imaging the larvae and classifying the larvae into at least one of two classes of male and female based on fluorescence detected, and placing individuals of one of the classes into a container designated for the respective class.

According to a third aspect of the invention there is provided apparatus for rearing of aquatic insects, comprising:
a rack;
at least one water tray, the tray having an interior tray surface and being fillable with water to provide a water surface; and
a robot, the robot configured to push the tray to one side of the rack for feeding and to a second side of the rack to tip said tray to empty said aquatic insects into a collecting area.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified diagram of a separation apparatus for imaging aquatic insects according to a first embodiment of the present invention;

FIGS. 2A and 2B are two diagrams of racks holding rearing trays for aquatic insects according to an embodiment of the present invention;

FIG. 3 is a simplified diagram of a tray for rearing aquatic insects according to an embodiment of the present invention;

FIG. 4 is a view of the tray of FIG. 3 with a robot for tending the tray according to an embodiment of the present invention;

FIGS. 5A and 5B are views of the robot of FIG. 4 according to an embodiment of the present invention;

FIGS. 6A to 6C are views showing stages in a cleaning procedure of the tray according to an embodiment of the present invention;

FIGS. 7A and 7B are views showing stages in a cleaning procedure of the tray of FIG. 6 according to an embodiment of the present invention;

FIG. 8 is a further view showing a stage in the cleaning procedure of the tray of FIG. 6 according to an embodiment of the present invention;

FIG. 9 is a detail showing two positions of the mesh during cleaning according to an embodiment of the present invention;

FIG. 10 is a view showing water inlets and outlets in a tray according to an embodiment of the present invention;

FIG. 11 illustrates a cleaning station for a tray according to an embodiment of the present invention which may be incorporated into a robot for attending a tray;

FIGS. 12A and 12B are views showing shutters on a rearing tray according to an embodiment of the present invention;

FIG. 13 is a flow chart showing a cleaning procedure according to embodiments of the present invention;

FIG. 14 shows trays in a stack with their outlets connected according to an embodiment of the present invention;

FIG. 15A illustrates sensors for use with the trays in a stack according to an embodiment of the present invention;

FIGS. 15B to 15D illustrate connections for larva inlets according to an embodiment of the present invention;

FIG. 16 illustrates motorized elements for a rack according to an embodiment of the present invention;

FIG. 17 illustrates an alternative arrangement for motorized elements according to embodiments of the present invention;

FIGS. 18A and 18B illustrate tube-shaped rearing trays according to an embodiment of the present invention;

FIG. 19 is a simplified flow chart illustrating a flow chart for an alternative cleaning procedure for a rearing tray according to an embodiment of the present invention;

FIG. 20 illustrates an alternative cleaning procedure for a tube-shaped rearing tray according to an embodiment of the present invention;

FIGS. 21A and 21B illustrate two views of the funnel entrance to the conveying area according to an embodiment of the present invention;

FIGS. 22, 23, 24, 25, 26, 27, 28 are views of various details of the funneling and counting mechanism according to embodiments of the present invention;

FIGS. 29A-29B, 30, 31A-31D, 32A-32B are views of images that require classification according to embodiments of the present invention;

FIGS. 33A-33B, 34A-34B, 35, 36, 37, 38, 39, 40A-40E are various views of the funneling and counting apparatus according to various embodiments of the present invention;

FIGS. 41A and 41B are views of structure that may be added to a rearing tray for aquatic insects according to embodiments of the present invention; and

FIGS. 42A-42B, 43, 44A-44B, 45, 46A-46D, 47, 48A-48B are views of various embodiments of a rack for holding rearing trays according to further embodiments of the present invention.

### DESCRIPTION OF SPECIFIC EMBODIMENTS OF THE INVENTION

The present invention, in some embodiments thereof, relates to constructions, systems and methods for automated or semi-automated rearing sorting and counting of pupae and larvae and, more particularly, but not exclusively, to rearing, sorting and counting of mosquitoes.

In one embodiment, a system and a method for automated rearing of larva is presented.

The embodiment may provide a system that enables controlled feeding, homogenous distribution of the food, and further may support repeated and controlled cleaning of the water trays to support high water quality for the growing of the larva. Embodiments may incorporate the ability to measure different parameters of the water and/or larva as they grow in order to fine tune the automated treatment or feeding, they receive.

One of the risks when rearing larva is that during rearing, the water may start accumulating bacteria and waste products which are harmful to the larva, resulting in death of the larva colony within the rearing tray.

The present embodiments may minimize such risks by occasionally cleaning the water automatically without taking the larva out of the water tray. The combined cleaning and feeding is aimed at maximizing the chances that all or a majority of the larva are fed and that a minimum of food remains in the water uneaten, thereby removing a potential source for growing bacteria and fungus. Cleaning may be carried out before a new feeding cycle or at a defined time after a feeding cycle.

A rearing unit according to the present embodiments may include a rack carrying multiple water trays.

Each water tray may consist of a mesh surface inside the water tray. The water tray may be rounded, tube shape, square, rectangular, or of any other suitable shape.

The larvae are set to grow in the water of the tray above the mesh.

In embodiments, every time it is required to change the water, the mesh is raised into the air just above the water level, allowing free passage of water underneath. Then water flows below the mesh and cleans the water tray. Outlets such as outlet valves and shutters may be opened in order to enable flow of water out of the tray. Preferably, at the same time, insertion of water from an inlet into the water tray keeps the tray from drying, so that after the dirty water is removed, clean water provides an immediate replacement.

Once finished, the mesh is lowered to be submerged inside the water, and the larvae continue growing inside the tray. When the larvae become pupae, they are flushed out of the water tray.

The present embodiments may provide an ability to flush out different trays following a trigger, unlike existing larva rearing racks which only enable the flushing of all trays at once. Since there may be a difference in the growing stage between trays, it may be required to flush out one tray at a certain time of the day, for example in the morning, others at noon and the others at the evening, to maximize the yield of each tray (number of larva that became pupae), because larvae in different trays may pupate at different times.

During the growth stage, a mobile tray manager unit moves along the tray and injects materials into the water tray: new larva, food, water. By traveling along the tray it supports a homogenous distribution of such materials.

The aim of moving the mesh up and down is to change the height of the mesh surface relative to the water level. Alternative embodiments enable the mesh surface to be fixed in place while changing the water level relative to the mesh surface by changing the water level height.

Embodiments may accordingly provide the ability to control the feeding and larva extraction separately from each tray, and an ability to manage the extraction of estimated amount of larva from each tray without another machine that separate and count larva. A rearing unit of the present embodiments may include a type of rack suitable for holding multiple larva water trays which are able to be pulled out, and also able may have their content removed separately, e.g. by pushing forward, as the tray is pushed on a slope so the water and larva spill out, or by tilting or other as described in greater detail herein.

A rearing robot may travel across or up down and sideways over the larva trays, and may be equipped with: [1] an arm to pull the larva, [2] an arm (can be the same) to push the larva tray, and [3] a food dispenser.

With the present embodiments it may be possible to automatically synchronize between estimated pupation time of pupa according to their location in the rack, say specific tray raw/height, and the flushing of this tray, in order to maximize the overall number of pupae being flushed daily from the entire rack or the rearing system.

Embodiments may provide a unique larvae tray design to support a high density of larva in each tray.

Embodiments described hereinbelow may solve the problem of counting and transferring a specific number of pupae and/or larva from a water container into another container. Embodiments described herein may also solve the problem of separating pupae from larva, as well as sorting male and female pupae, providing many advantages associated with the solution.

In further embodiments, combined larva separation and counting is provided as well.

In an embodiment, a water container contains the insects in their aquatic phase, such as mosquito larva or mosquito pupae. A mechanism enables propagation of the insects out of the container and towards a conveying mechanism. In such an embodiment the conveying mechanism may be a conveyor with a permeable conveyor belt. That is to say the belt may be permeable or porous, the terms being used interchangeably herein to refer to material with holes or mesh or which are net like, enabling the exchange or passage through or leakage of liquid across the belt.

The porous conveyor enables flushing the water down through the conveyor mesh as the conveyor passes from a first end to a second end, while retaining the insects on the conveyor belt surface.

The conveyor may propagate the insects towards an imaging location. At the imaging location, a camera may obtain images of the insects propagating on the surface of the belt. An electronic classifier, for example an image processing algorithm, may use the obtained images to identify the objects moving on the conveyor as either individual pupae or larva. In an embodiment, the classifier may distinguish male pupae from female pupa, or may distinguish pupae from larvae, or may distinguish between pupae or larva on the one hand and another class of all objects which are not pupa or larva. The expert may implement any possible combination for identification based on that concept.

Once an object is identified, software may update a counter corresponding to the class identified, for example the same group of insects. Herein the terms classifier and identifier are used interchangeably referring to classification and/or identification of types of insects in an image unless the functions of classification and the function of identification are described explicitly as two different steps. Any given image may contain more than a single insect, which can then be cropped and then multiple crops may be sent to the classifier as multiple images, or as a single image with multiple insects on it which the classifier may then detect and classify automatically.

In embodiments, the conveyor may continue propagating the insects after the image is obtained, thereby to place the now classified, or being classified, insects in the water of a receiving container. Thus, after completing the counting process, the counted insects are transferred into water in a container so they can be removed. Other embodiments for transfer of the insects into a receiving container following classification are discussed hereinbelow and may include water jets.

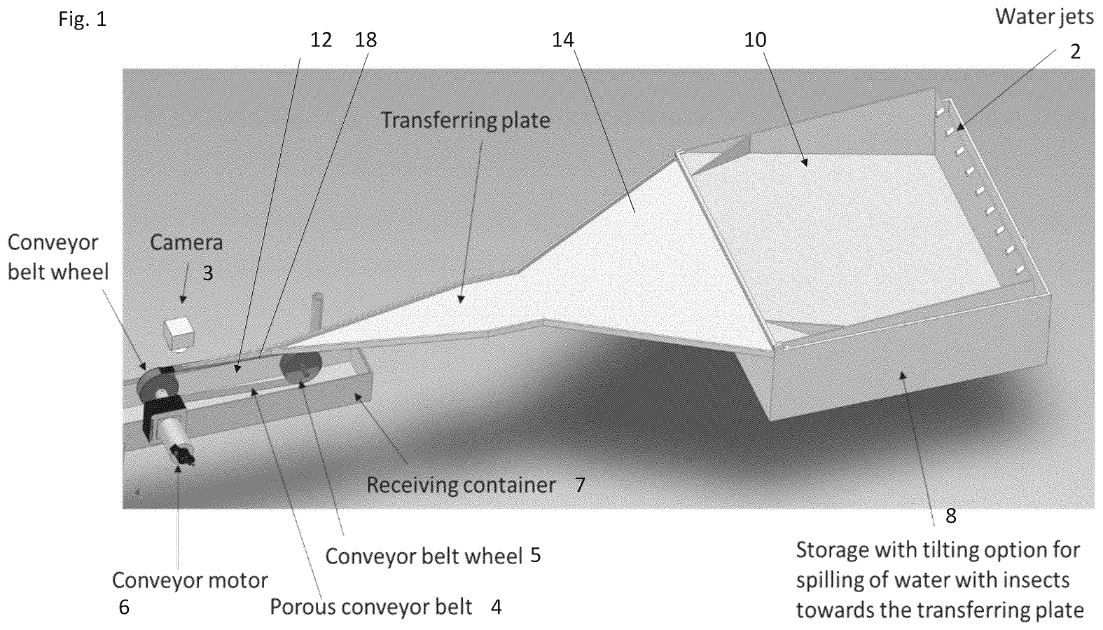

Reference is now made to FIG. 1, which illustrates a construction for handling of larvae. The drawing is more fully described hereinbelow.

Insects in the form of larvae or pupae may be transferred from a storage container 10 to a receiving container 12 via a transfer plate 14. The insects may be aquatic pupae and/or aquatic larva, such as for example mosquito pupa and/or mosquito larva. The pupae or larvae may be only males or only females or both males and females.

From the transfer plate 14 the insects are directed to conveyor belt 16 which includes a permeable surface. The belt surface may be partially permeable or entirely permeable and the permeable surface may enable leakage of water while retaining the insects on it.

Directing of the insects to the transfer plate 14 and conveyor belt 18 may include using a controlled directing mechanism. Such a mechanism may include an actuator which tilts the content of the insect storage container 10 towards the permeable surface 18 so the water with insects is poured out of the water container and towards the permeable surface, or towards the transfer plate 14 which directs the water with the insects towards the permeable surface 18. Controlled directing may be fulfilled by other means such as pushing water away from a water container by utilizing a piston, or by suction of water using a valve from the water container towards the entry position onto the permeable conveying surface 18.

The insects are conveyed on the porous surface. Additional embodiments are described later on such as for example a rotational disc.

The insects may be imaged using camera 20 as they pass on the porous surface of the conveyor belt 18, and imaging may be carried out in the visual part of the spectrum or at non-visual wavelengths. Insects may be fluorescent, meaning they emit light of a specific wavelength upon excitation using the same or another specific wavelength.

Imaging may include use of a light source which is capable of emitting the required wavelength to excite a fluorescence response from the insects and the camera may be able to identify the emitted fluorescence light, which, as said, may be of a different wavelength.

Then insects may be electronically classified from said images into classifications including male mosquito pupae, female mosquito pupae, mosquito pupae, mosquito larva, male mosquito larva, female mosquito larva, colored larva and unclassified objects.

Classification may include classification of insects according to identification of emitted fluorescence color, which information may be combined with identification that the emission comes from a specific larva object or a specific larva body part.

Classifying may be carried out using a trained neural network, and the neural network may be a classical neural network or a deep network, including a convolutional neural network, or any other kind of neural network. The trained neural network may comprise four or more layers.

The detection, namely the task of finding the insects in the image, and classification, the task of determining say which gender type it is, for example male or female pupae, may be jointly solved using known algorithms such as YOLO, Faster RCNN and others.

Various operations may be applied to the insects, such as transfer of the insect into a receiving container by means of propagating the conveyor belt into the water, and letting the insects detach or swim away from the belt, or be taken away using a suction element or being destroyed using a suction device or a laser, or taken away using a water jet or a blower device.

An advantage of some embodiments is an ability to control the velocity of the insects at the imaging location. The velocity of the carrying surface (e.g. the permeable conveyor belt) may change between higher than zero to a complete stop, therefore controlling the velocity of the insects located on it, with the ability to have the belt and the insect on it stop below the imaging sensor (e.g. camera) for the purpose of imaging the insect.

The insects are thus moved into a thin volume, funneled to go one by one, and are then imaged, for counting, classification etc. In embodiments, larvae may move inside water pipes, and imaged and the unwanted insects, usually females, may be destroyed by laser while within the pipes.

The following is a description of an embodiment with the ability to change the velocity of the insects:
Obtaining insects inside a water container 10;
Directing the insects to a permeable surface 18;
Conveying the insects on the permeable surface 18;
Conveying may be implemented by having a conveyor and having the conveyor belt as the permeable or partially permeable surface.
Controlling the velocity of the permeable surface;
Controlling the velocity may involve changing the velocity between a complete stop of the permeable surface and any velocity higher than zero. An electronic switch may switch between a stop and higher than zero speed of the permeable surface. The switch may be a hardware switch or implemented in software to command the conveying system to stop moving.

The permeable surface may be partially permeable. The permeable surface may be a porous belt.
The insects may be imaged.
Using the images the insects may be classified using a classifier.
An operation may then be carried out on the insects;
The operation may comprise transfer of the insects into a water container by moving the conveyor belt into such water container. The insects may be enabled to swim away and detach from the conveyor belt, or some kind of extraction of individual insects from the conveyor belt may be provided on the basis of the classifier results. Extraction may for example use controlled water jets or a blower device or a suction device to extract individual classified insects.
Classification using a classifier may classify the insects into pupae and non-pupae, or male pupae and female pupae, or male pupae and female pupae and ‘other’, or larva and non-larva, or pupa and larva, or male pupa and female pupa and larva, or male pupa and female pupa and larva and ‘other’, or male larva and other or male larva and female larva and other or female larva and other.

The present embodiments may provide the ability to image the insects on the conveying device in good quality, because the belt is a permeable belt. This supports the separation between the water and the aquatic objects located within the water container and the belt surface. The separation separates the water from the insects while not harming the insects so that the insects are imaged on the surface and not through a thickness of water.

The following describes an embodiment for counting and/or separation and/or classification of aquatic objects by separating the objects.

Water with aquatic insects in a water container, such as mosquito pupa and/or mosquito larva is obtained.

The water is extracted together with individual aquatic insects towards a conveying water separation mechanism.

Use is made of the conveying water separation mechanism to separate between the water and the insects, thereby obtaining aquatic insects without water.

The water separation mechanism may help to convey or thrust the insects towards an imaging location.

Insects are imaged at the imaging location without layers of water intervening between the camera and the insect.

An electronic classifier may count and/or classify the insects into different groups.

In another embodiment an additional step includes separating insects according to the results of the classifier, for example depending on the classification either conveying the insect back into the water or separating it out mechanically, for example using a water jet or air jet or suction device or blower device.

The solution may be applied to other insects and even more generally objects which are present in water and which can be carried by the water as the water is extracted towards a separation mechanism.

In an embodiment the conveyable permeable surface is submerged within the insect water container. The conveyable permeable surface conveys the insects from the water container towards an imaging location. As the conveyable permeable surface emerges from the water, the water on the permeable surface is flushed downwards and the insects remain on top, being propelled towards the imaging location where counting, classification and possibly sorting and transfer are implemented as described elsewhere herein. In an embodiment, the water level inside the water container is being lowered to support the conveyor operation to collect the insects and transfer them towards the imaging location. In an embodiment, water jets may push or propel insects towards the submerged permeable conveying surface, to collect and convey the insects towards an imaging location for counting and/or classification and/or sorting. At the imaging location an electronic classifier may classify the insects as discussed.

In an embodiment the insect permeable conveying mechanism comprises a linear conveyor belt.

Another advantage of separating the water and insects, and extracting the water, is that the material carrying the insects is no longer the water, but a solid surface (the porous belt for example) on which they are located and conveyed. Hence the method may change the carrier material from a liquid carrier (the water) to a solid carrier (the conveying surface).

An advantage of carrying the insects on a solid surface as in embodiments of the present invention is that the speed of movement of the belt becomes the speed of the carried insects and may be controlled by controlling the speed of the belt, say via the rotation speed of the conveyor motor. Controlling the speed of the insects travelling in water by controlling the water velocity is more challenging, since turbulence may ensure that there is no uniform velocity of the water.

The location of the insects on a solid surface ensures that the insects are more easily viewed and imaged, as mentioned above. The embodiments may enable separation between the aquatic insects and the water without harming the insect.

A further embodiment is now described.

Objects inside a water container are obtained.

The water containing the objects is spilled towards a porous conveyable area, which may be either completely or partly porous.

The water then drains or leaks through the porous material, thereby performing separation between the insects and the conveyable area.

The separated objects on the conveyable area are taken towards an imaging location, where each object is identified and counted, thereby counting the objects, typically insects at one life stage or another, that were in the water container.

The insects may then be classified into different categories and either left alone mechanically extracted according to their classification, thereby performing insect sorting. Mechanical extraction may for example be implemented using suction or a blower or a water jet.

The mechanical suction may use a moving suction arm that sucks insects from multiple coordinates on the surface as obtained from the camera imaging the insects.

The solution may combine efficient separation of the aquatic object from the water, while also conveying the aquatic object, preferably in horizontal orientation towards an imaging location.

A porous belt is one possibility in the described embodiments to separate water and aquatic objects and moving the objects.

Another possibility for separation of the aquatic objects from water and conveying them may be achieved by creating a conveyor-like structure in which the carrying material is made of a series of discs with gaps in between them that allows water to bridge the gaps between the discs, but the aquatic objects, be they larva and/or pupa, remain on the surface of the discs. Such typical distance between the discs would be lower than 0.5 mm preventing larva getting caught in between the discs.

Connecting together all the discs and creating a belt-like shape made of discs, may enable propagating the insects by propagating the discs they are located on, while flushing water through the gaps between the discs.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

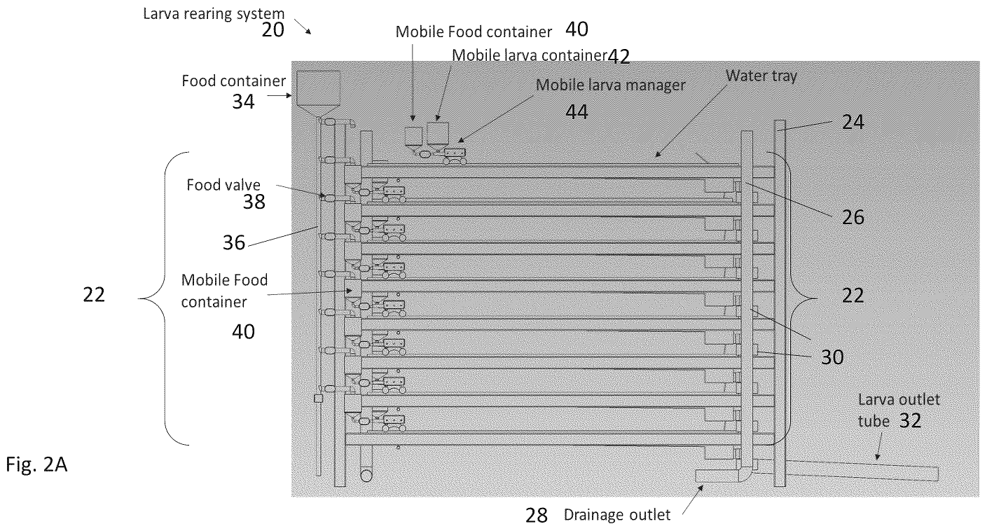

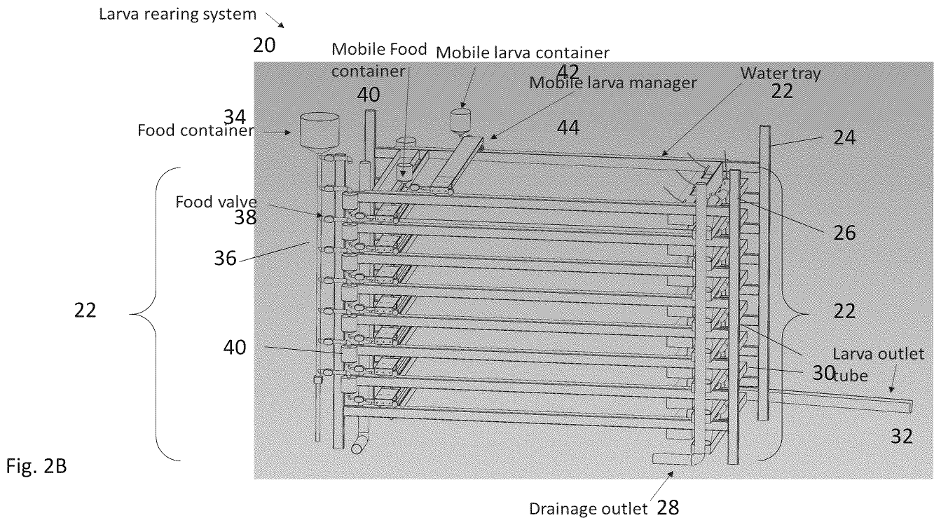

Reference is now made to FIGS. 2A and 2B which illustrate a rearing unit for rearing larvae and pupae according to an embodiment of the present invention. The two figures are the same except that the larva outlet tube is in a different position as will be explained.

Rearing unit 20 includes stacked water trays 22, a frame 24 holding the water trays, a dirty water drainage pipe or pipes coming out from each water tray and leading to a drainage connecting pipe 26 and drainage outlet 28, and a larva outlet pipe or pipes 30 coming out from each water tray to any one of which is connected a larva outlet tube 32. In FIG. 2A the larva outlet tube 32 is connected to the second lowest tray and in FIG. 2B it is connected to the lowest tray.

A food container 34 leads to a dispensing pipe 36, from which valves 38 allow food to be dispensed to individual trays 22. The pipe and valve system form at least part of a mobile larva manager and each tray has a mobile food container 40 between the valve and the tray. Food dispensing is managed for individual trays by selective operation of the valves.

A mobile larva container 42 is also connected to the mobile larva manager.

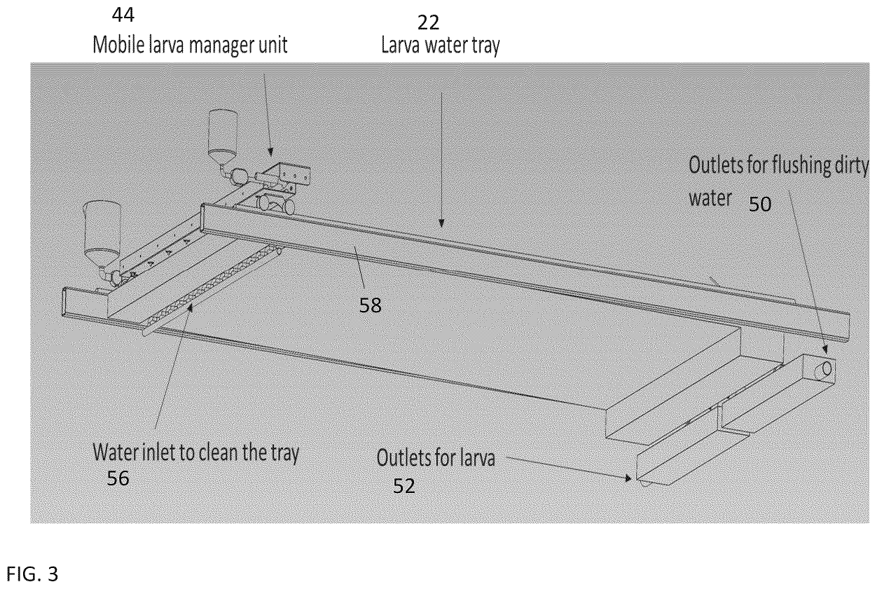

A mobile larva management unit 44 includes the mobile larva container, mobile food container and pipes with nozzles to direct and drop (preferably liquid) food and/or larva and/or water as it travels along the water tray according to commands provided by a software controller. Reference is now made to FIG. 3, which illustrates an individual tray 22 with outlets 50 and 52, the mobile larva manager unit 44 and water inlet 56.

The water tray 22 includes a frame 58 for holding the tray, a permeable surface, for example a mesh, which can be lowered and raised between at least two positions, that is up against the water inlets and outlets.

In the upper position, the water is flushed down through the mesh surface while the larva stays on top of the mesh surface given the mesh holes are smaller than the larvae typical size.

In the upper position water may enter the water tray from the water inlet and flow through the tray, and flush the water through the mesh. A trigger triggers the system to stop pouring water in, thus exchanging the water automatically within the larva water tray, and providing a cleaner environment for the larva to grow in. After exchanging the water, the mesh height may be adjusted relative to the water level so that the mesh is submerged once again in the water enabling the larva to swim and float.

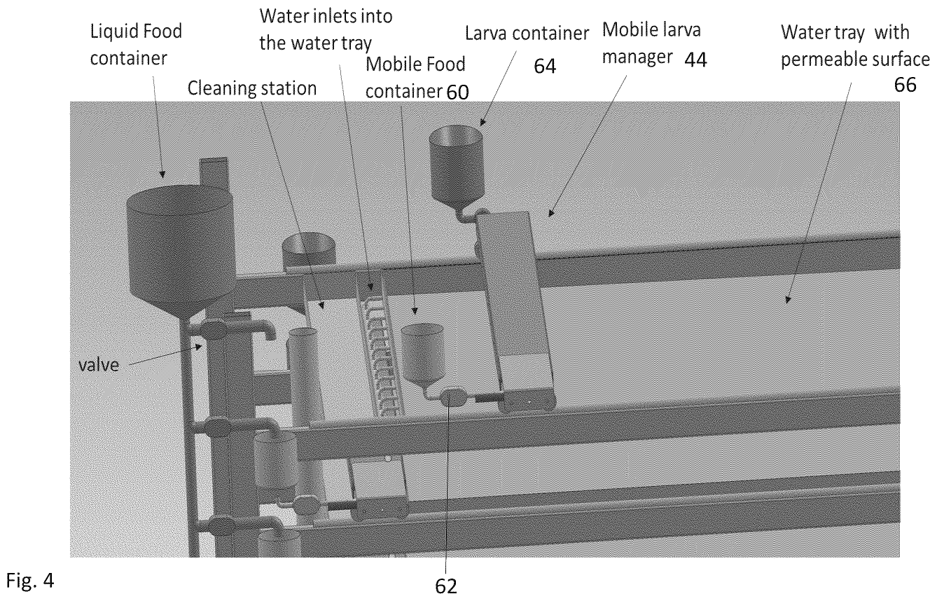

Reference is now made to FIG. 4, which shows in greater detail the mobile larva manager unit 44 as seen from above.

In embodiments food container and/or the mobile food container 60 comprises a vibrator device 62 to cause vibration and vibrate the water with the food before dispensing the food in order to distribute the food homogenously within the food container. In an embodiment instead of vibration there is a rotational element that rotates inside the water in order to distribute the food homogenously within the food container 60.

In an embodiment the larva container 64 includes an external or internal vibrator or internal rotation element to vibrate or spin or rotate or move or swirl the larva to be homogenously distributed within the larva container 64 in order to support even dispensing of the larva into the water tray 66 in order to prevent a situation in which larva are unevenly distributed around the tray.

In an embodiment the water tray is divided into multiple cells. Each cell may be of the same shape and size or they may be different sizes. A cell may be a square shape or long rectangles or other. Dividing the water tray into cells may allow the homogenous distribution of the larvae within the larvae water tray, and dispensing the larvae in each cell. Later dispensing of food within each cell may correspond to the estimated number of larvae within that cell.

The manager unit functions as a mobile unit that travels along the water tray and dispenses materials according to need. The manager unit may dispense larva after initial filling of the larva container. By traveling along the water tray, the unit may dispense larva homogenously through the unit nozzles, along the tray. The unit may also dispense food such as liquid food from mobile food container 60, and may also dispense water.

As the mobile unit travels and dispenses food, it may ensure the food is distributed homogenously along the water tray.

Also, under software control, the mobile unit may traverse the water tray 66 multiple times per day and dispense small amounts of food per each time, e.g. some set daily amount divided by the number of times it dispenses food per day.

Such a method of distribution may increase the chance of having cleaner water, and reduces the chance of food leftovers remaining in the water.

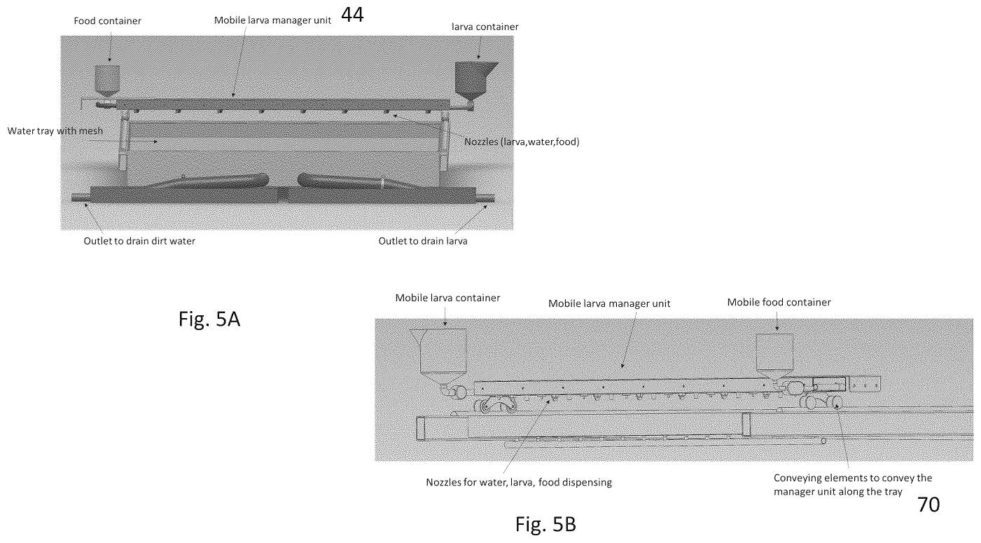

Reference is now made to FIGS. 5A and 5B which illustrate the larva management unit 44 and a travel mechanism for moving along the water tray.

The mobile larva manager unit 44 includes conveying elements. In an embodiment the conveying elements are wheels 70 providing the unit the ability to smoothly travel along the tray.

The unit may be motorized, traveling on its own along the tray, or it can be passive, being connected to a moving element which conveys it along the tray.

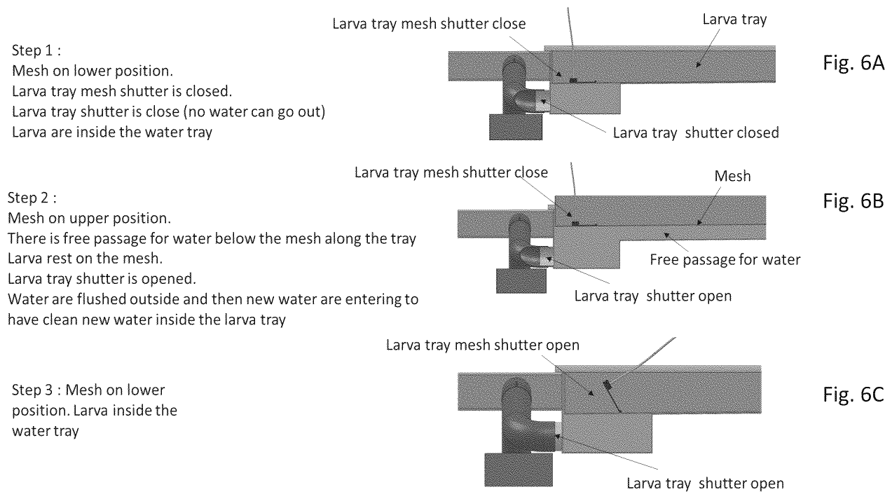

Reference is now made to FIG. 6, which illustrates a three-part method for keeping the water inside the trays as clean as possible. There is an advantage in the ability to quickly change the water inside the tray without harming the larva.

The water tray includes a mesh which may be lifted with the larva on it. Normally the mesh is at the bottom of the tray, as in FIG. 6A.

As the mesh is lifted above the water level, the water remains in place while the larva are lifted with the mesh surface—FIG. 6B. The larva tray shutter may then be opened and the water exchanged for clean water. That is to say a cleaning process involves opening the Larva tray mesh shutter to allow flushing of the water while the larvae are held safely on the mesh.

Fresh water may then enter from the tray water inlets and flush and clean the tray from side to side. The exact location of the water nozzles and their shape may vary, e.g on various sides of the tray, with larger or smaller nozzles and with different shape etc.

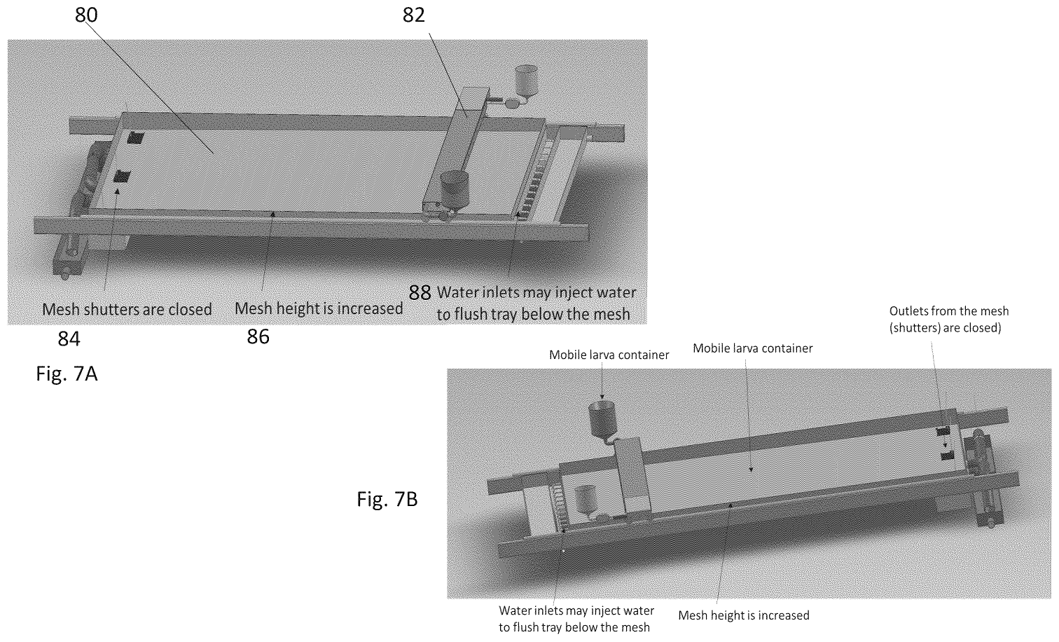

FIGS. 7A and 7B illustrate two different views of a water tray 80 with mobile station 82, mesh shutters 84 closed, and the mesh 86 at its upper position, allowing water inlets 88 to inject clean water to fill the tray and flush out the dirty water.

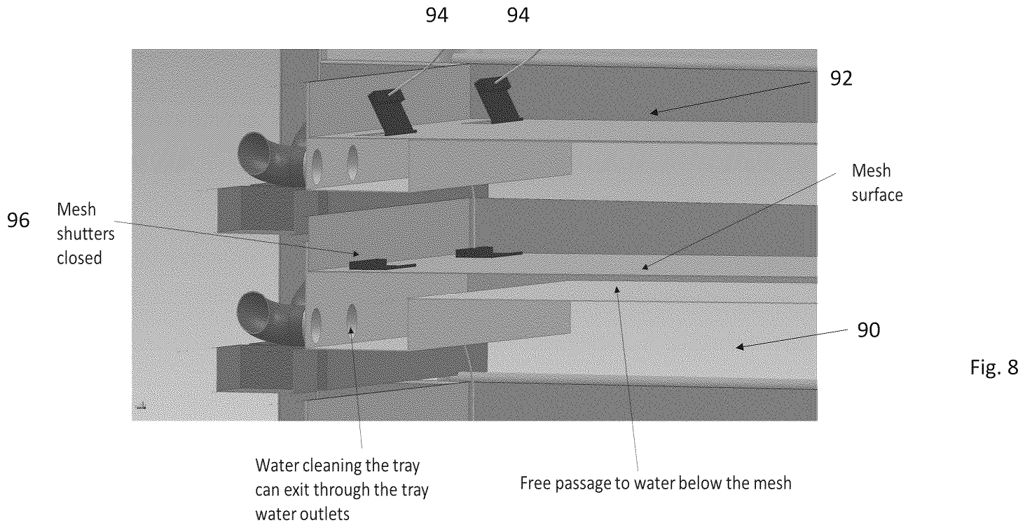

Reference is now made to FIG. 8 which shows a cross section with two trays 90 and 92 one over the other. The lower tray 90 has the mesh in the upper position, with shutters 94 open, the allowing the cleaning of the tray without harming the larva. After cleaning is over (e.g after a predetermined time), the mesh surface is lowered to its lower position. The upper tray 92 has the mesh in the lower position and the shutters 96 are closed.

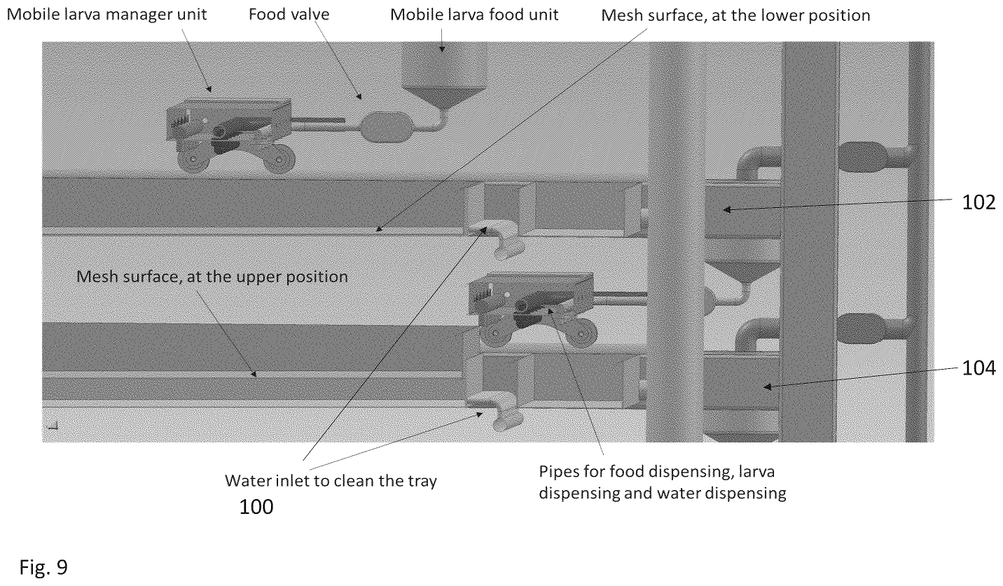

Reference is now made to FIG. 9, which is a simplified cross sectional drawing showing the water inlets 100 in greater detail. The upper tray 102 has the mesh in the lower position and the lower tray 104 has the mesh at the upper position allowing the cleaning of the tray.

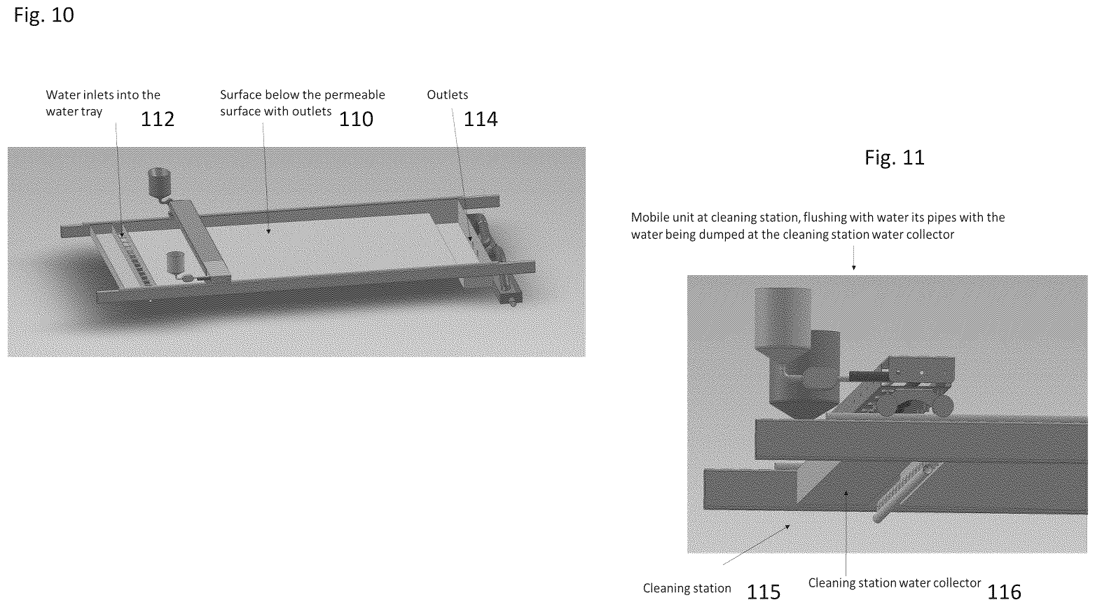

Reference is now made to FIG. 10, which is a simplified diagram showing a water tray according to the present embodiments without the mesh surface to better show the water flow. The water flows across tray surface 110 from water inlets 112 towards outlets 114 at a second end. The water may thus be flushed through during the cleaning process and may travel below the raised mesh thus not flushing out the larvae. The water flushing process may be controlled by a cleaning station 115, and the water may be collected at a water collector 116—see FIG. 11.

Regular cleaning of the water may avoid leftovers (e.g. food) from the previous day remaining in the water tray. Rather, according to the present embodiments, each day, or multiple times a day, the water can be replaced ensuring no food leftovers remain in the water, minimizing any risk of contaminating the water with bacteria and/or other harmful components.

In an embodiment cleaning is performed independently at each of the trays in the rearing rack. In another embodiment cleaning is performed at several or all of the water trays together.

In an embodiment larvae are transferred between the water trays, and cleaning, via lifting the mesh surface and changing the water as discussed above, is performed only at selected trays. For example a particular tray may be monitored and, once the majority of the larva have pupated the remaining larvae and dirt may be flushed away before collecting the pupae.

In an embodiment there are two water outlets from each tray, one for removal of the dirty water after a cleaning process, and another for conveying the larvae together with the clean water when the mesh is in its lowered position. There may be two types of inlets with multiple physical outlets per type. Thus there may be more than one outlet for conveying dirty water and more than one outlet for conveying larvae.

In another embodiment, there is a single type of outlet, which may include more than one physical outlet. At some location along the removal pipe, a valve may direct water to a different secondary pipe, depending on whether the material flowing away from the rearing rack is considered dirty water or is considered as useful, thus water with pupae or water with larvae.

In an embodiment the mobile manager unit does not include any water pipes, but rather only food and larva dispensing pipes, and all cleaning is only accomplished by activating the water inlets of the water tray.

In another embodiment, the water inlets of the tray do not exist or are not active and the mobile unit may include water outlets which pour water being flushed through the mesh and play a part in cleaning the water tray below the mesh.

In another embodiment both the water inlets of the water tray and the water outlets of the mobile manager unit are present and help to flush water when needed.

The feeding may be in several sessions distributed over the day as mentioned, with small doses per feeding event. The food may mainly consist of fish powder.

When providing food once a day, there is a risk that if there is too much food, harmful bacteria and germs will start developing in the water, and by distributing the feeding into small doses along the entire day, the risk is much lower.

The larvae grow inside the tray and daily receive an amount of food required for that stage in their life, preferably multiple times per day as mentioned.

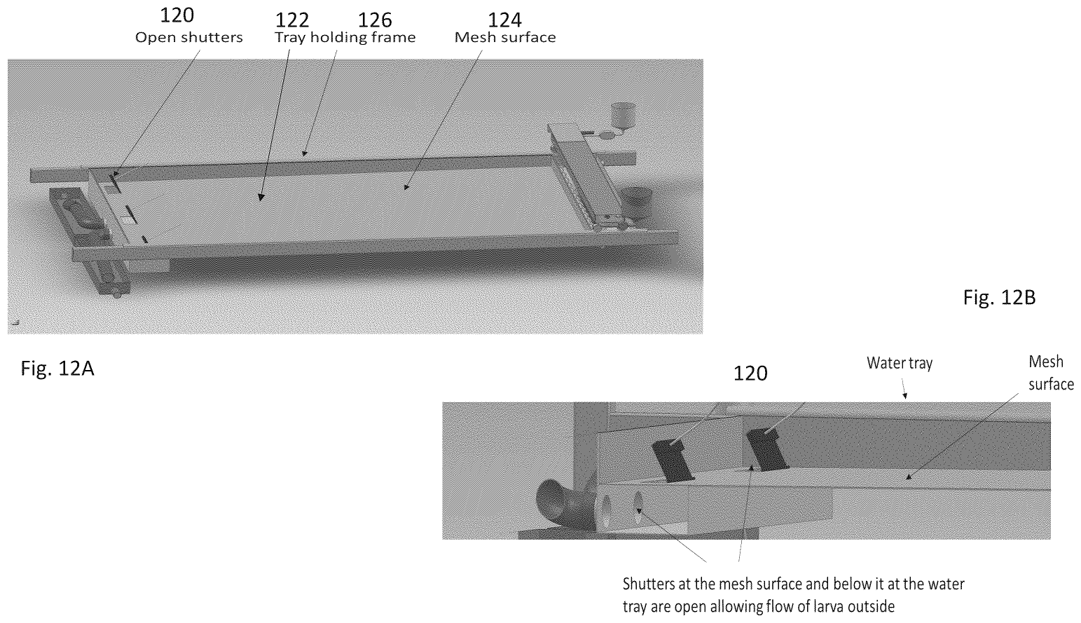

When it is time to extract the larvae or the pupae from the trays, then as shown in FIGS. 12A and 12B, mesh surface shutters 120 are opened while the mesh surface 122 is at its lower position on the surface of tray 124, and the larvae can flow out.

While drawn as a group of three square holes, the shutter may be of any shape and size and may also for example be one large hole extending along the entire tray width being opened and closed as needed. Alternatively, the shutter may be a single opening with the water tray surface having a slope to direct the water and larvae and/or pupae to flow towards that hole.

In the above description the cleaning process includes lifting the mesh surface upwards and after flushing the dirty water and injecting clean water, lowering it back into place. The mesh is preferably held by a holding frame 126, and the frame itself may be lowered and raised when lifting and lowering the mesh. Lifting the mesh may be performed by lifting the entire mesh at once from multiple sides, or by lifting one side first and then another other side. Lifting and lowering refers to the action of bringing the mesh to a position above the water level so the larvae will be out of the water and resting on top of the mesh, so the water tray below may be flushed. Nozzles at one side or multiple sides along and/or around the water tray frame may inject water, or there may be only a single water inlet to flush the entire water tray. The water tray surface may include a slope such that water may always travel in one direction, or towards the direction of the outlets to support a smooth water changing process. The mesh may be made of strong material such as aluminum mesh and may be supported at a number of points that may be lowered and lifted without the need to hold the entire mesh within a frame structure.

In an embodiment, the apparatus for automated rearing of larva include at least a rack of water trays, and a set of larva mobile manager units.

Thus, a larva rearing water tray may comprise: a larva outlet, a drainage outlet, a surface bounded by surrounding walls to hold water, a mesh and preferably a mesh holder. Herein, when referring to lifting or lowering the mesh, reference may be understood to relate to a frame holding the mesh which is being lifted or lowered within the area of the water tray. Alternatively, the mesh may be fixed and the water level may be changed. The tray may further include a mechanism to change the mesh height relative to the water level, or change the water level relative to the mesh height). Shutters may enable flow of water with larva or pupae as appropriate from the larva tray while the mesh is in the low position, enabling flow of the larva with water outside the tray. When the mesh is in the upper position, so that a majority or all of the larva rest on top, water may be changed below.

In an embodiment changing the relative height of the mesh surface to the water is performed by changing the height of the mesh by physically moving the mesh itself up or down, or by rotating the entire water tray upside down and flipping between a situation where the mesh is below the water level and above the water level, say by flipping or rotating on its axis, or by lowering and increasing the water level which is then below or above the mesh surface height. The height may be further controlled by flushing out water when the outlets are opened or by closing the outlets and adding water.

In an embodiment the water tray includes one type of outlet, so called water outlets.

A conveying mechanism to lower and lift a frame holding a mesh may include one or more motors and a belt commonly used to convey elements, or a piston or multiple pistons used to move the frame up and down or other common methods used to convey an element back and forth between two or more positions. When only the water level is changed, a conveying mechanism to change the position of the mesh may not be required. Shutters may be implemented for example by the introduction of valves, shutters, electronic valves, hydraulic valves or the like.

Mobile unit 44, referred to herein as “manager”, “unit”, “larva manager”, “larva mobile manager unit”, “larva manager unit”, “manager unit” or similar may traverse actively or passively along a larva rearing water tray water tray. As explained, the unit may include one or more of a food dispensing pipe and nozzles or orifices connected to a food container or a mobile food container, a water pipe and nozzles connected to water valve or water container, larva dispensing nozzles and pipe connected to a larva container or a mobile larva container. The unit may further include a sensor or multiple sensors providing parameters on the water quality, e.g. camera, odor sensor, ultrasonic sensor, proximity sensor. Other sensors may provide data on the status of the larva such as size, number, color, etc. and other sensors may identify the water level to identify if additional water is required.

In another embodiment the rack of water trays does not include the cleaning mechanism to change the water but does include the mobile manage units, while another embodiment may include the cleaning mechanism to change the water per each or some of the water trays as described above, but does not include the mobile larva manager units.

A rack may include multiple larva rearing water trays and multiple mobile manager units as described, to support automating the process of larva dispensing and/or larva feeding and/or larva counting and/or changing water.

The system may include a homing position associated with the mobile unit. The system may include a loading position where the mobile manager unit and associated mobile food container are being filled with food from a main food container which is able to distribute food to all mobile units via controlled valves which dispense the food into each mobile unit. The mobile unit may be filled with larva at a location associated with the homing position or the loading position.

A controller controls the operation of the rearing unit, or multiple units together, using a software or firmware or like program. The controller may be any type of computer which can run software programs and may send commands to external units using communication protocols.

The controllers may be programed to determine at what intervals to feed the larvae, how much food to dispense per each cycle on the day and per each day, when to trigger a cleaning cycle and when to end the cleaning cycle, when to flush out larva and when to stop flushing them out, when to open valves and add water, etc. All parameters may be manually calibrated by setting different parameters for the software for example using a user machine interface to receive such calibration parameters from a technician.

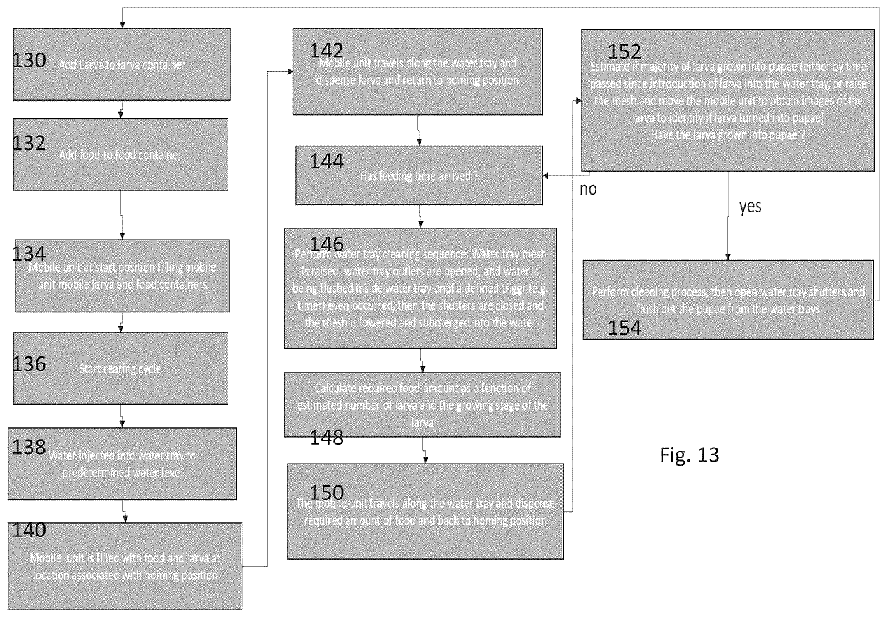

Reference is now made to FIG. 13, which is a simplified flow chart that illustrates operation of the automated rearing rack. In box 130, larvae are added to a larva container. In 132 food is added to a food container. In 134 the mobile unit is at the start position and food and larvae are transferred to the mobile unit. In 136 the rearing cycle begins. Water is injected into the tray to a preset level 138 and the mobile unit is at the start position 140. The mobile unit travels along the tray 142 and dispenses larvae evenly, for example using vibrations, and returns to the start position. The system checks for feeding time 144, and if so carries out a cleaning process 146 which involves raising the net, and changing the water, as described. In box 148 the amount of food needed is calculated, based on the number of larvae and their stage of development. In box 150 the unit dispenses the calculated amount of food, using vibration etc as necessary for even distribution. An estimate is made in box 152 as to whether the majority of larvae have pupated, and if so the tray is flushed one last time 154 to collect the pupae.

While the water trays shown in the drawings are square or rectangle, other shapes and forms of trays may be implemented such as circular or circle shapes, elliptic, tube shape, half-tube shapes or others. The mobile manager unit may move in a straight line above the water tray but may alternatively move in a circular shape following any radius and curvature of the water tray.

In an embodiment the water tray is of the shape of a tube with opening on its top for dispensing of food and larvae.

Water trays may be aluminum, plastic or of any other suitable material, the conditions including being lightweight, having sufficient mechanical toughness for the weight of water involved, being non-toxic to the insects and being waterproof.

The mobile manager unit may alternatively be viewed as a mobile movable bridge dispensing unit, but still having the same functionality as described herein. Dispensing has been described above as injecting the material from pipes through orifices and/or nozzles. In other embodiments other methods to convey materials from a mobile food container and/or mobile larva container may be implemented such as rotating screw (and other similar screw like shapes) which convey material such as food towards a target location.

The water inside the water trays may be warmed by warming the water larva trays and the mobile manager unit may also include one or more temperature thermostats to measure the water temperature at different areas. The mobile unit may include a heater to heat the water to a required temperature, for example 27 degree Celsius). In such embodiment, if the mobile unit measures a temperature which is below a required threshold it may heat the water in that area and may also trigger an alert.

In changing the water in the water trays process, it is possible to inject water at required temperature, and if required, different temperatures may be provided for each water tray for example depending on the height of the water tray. For example, for a given tray regular temperature may be used (e.g. 27 degree) while for another given tray the water may be set to 29 degree Celsius, for example to compensate for any possible drop in water temperature as a function of the height of the water tray. Once the temperature level dropped below a threshold and the event is detected, heating the water or heating the air surrounding the water or changing the water with hotter water is possible.

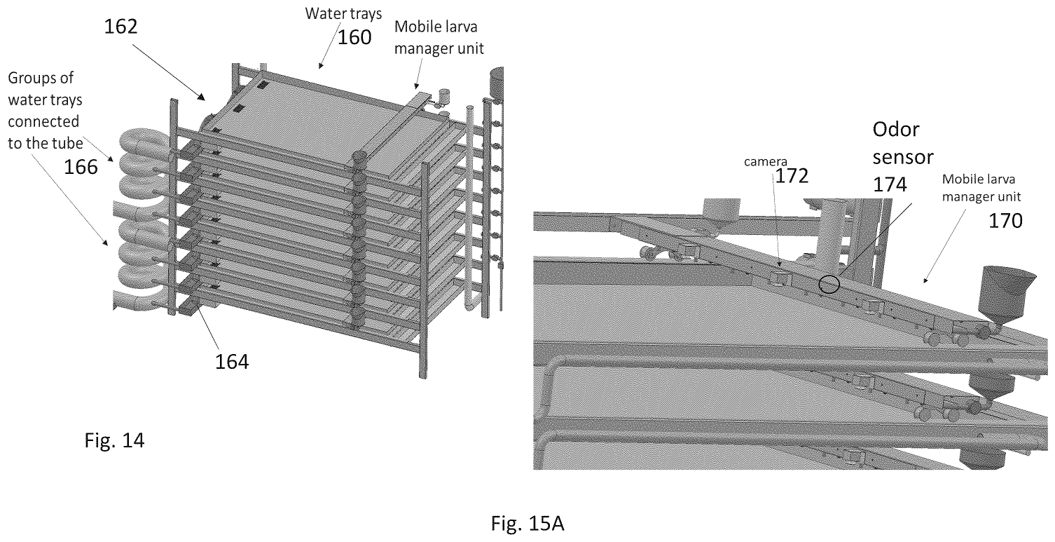

In an embodiment the mobile larva unit managers are all connected to a single conveying component, and in another embodiment the mobile larva unit managers are moving by their own. Reference is now made to FIG. 14, which is a simplified diagram of multiple water trays 160 in a stack 162. Two or more larva water trays outlets are connected together and their drainage water outlets 164 are connected together to a single outlet tube 166.

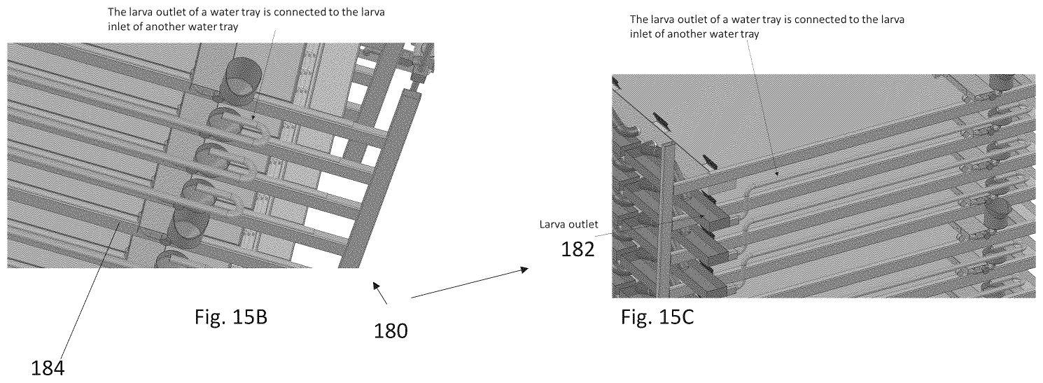

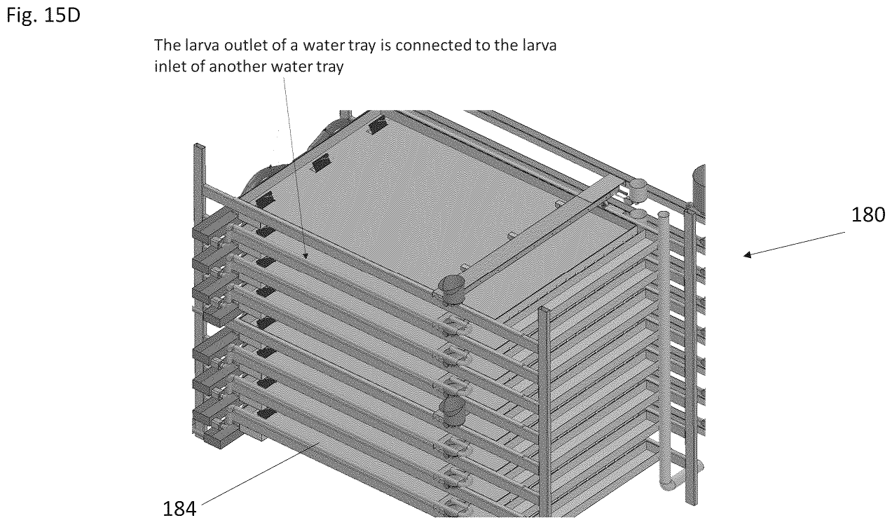

Referring now to FIG. 15A, the larva mobile manager unit 170 comprises an imaging sensor such as a camera 172. The sensor may detect large groups of larvae in the obtained images by an image analysis algorithm, which may trigger a different dose of food to be dispensed at the location or vicinity of the large group by the larva mobile manager unit. Detection of multiple objects above a clean surface, whether water or mesh, may be implemented for example by identifying the number of darker pixels, which representing objects on the water/mesh, relative to lighter pixels which represent the water or the mesh itself without other objects in or on it. The imaging sensor may also detect changes in the color of the water, and hence may trigger a cleaning sequence.

The imaging sensor may provide additional images of the insects. This can be performed by lifting upwards the mesh surface to its upper position above the water level (or by lowering the water level below the mesh surface), and then traversing along the tray, obtaining images of the mesh surface. Obtaining the images may be performed using a single camera with a wide field of view, or a single camera with a narrower field of view but which can also move along the mobile manager unit in an axis perpendicular to the main direction of movement. A further alternative may use multiple cameras to cover the entire mesh surface in each image. Following obtaining the images, common object detection algorithms may be applied to detect objects on the mesh and count the number of larvae on the surface. Neural network classification algorithms may be applied to identify and classify the larvae objects in the images and provide a more accurate count on the number of larvae on the mesh. Additional measurements may be applied such as measuring the size of the larvae. All such image analysis on the number of larvae and potentially additional parameters like size, can be applied to automate decisions on cleaning cycles and/or amount of food to be dispensed in the water. For example, according to the estimated number of larvae counted on the mesh, the amount of food to be dispensed on that day is then calculated. Obtaining the images may be independently practiced on individual trays or may be coordinated. The images may be used to identify when the larvae became pupa. A point when the majority of the larvae became pupa may be determined by detecting the ratio of pupae and larvae in the image obtained. Images may be obtained of objects on the surface of the mesh when the mesh is at the high position so that water does not substantially intervene between the camera and the objects being imaged. The use of automated detection from imaging may increase the productivity of the rearing system, to flush out the insects from the trays after the estimated or calculated number of pupae reaches a predefined threshold, instead of extracting the insects after a predefined time without identifying if a sufficient number of larvae have already pupated. For example, if there is a temperature difference between the water tray of the first floor of trays and the highest tray, it may happen that the majority of larvae on these two trays will pupate at different times, hence, it is preferred to be able to measure actual pupations rather than just rely on an estimated time.

In an embodiment an odor sensor 174 may sense the presence of gas in the air hinting there is too much food in the water which is not being eaten, and may trigger a cleaning process as described above.

The odor sensor may also trigger cleaning process or trigger other alerts upon sensing unusual concentrations of any type of gas. Upon sensing and analyzing such abnormalities a trigger for starting a cleaning process may be triggered, or technician alerts may be triggered.

Reference is now made to FIGS. 15B to 15D. The trays are provided in a stack 180 as before and the larva outlet 182 of each tray 184 is connected to the inlet of the next larva tray, and the outlet of the last tray is then connected to a pipe or other means to collect all the water from all the tray outlets.

In the present embodiment, in each tray there may be a mesh of a different mesh size, wherein the first tray may include the mesh of the finest size. After the larva grow, and molt they turned into another larva instar with a larger size, and indeed mosquito larvae go through 4 instars from L1 to L4. Thus at each stage, the new larvae instars may be flushed in a cleaning stage to the next water tray with mesh larger than the previous mesh holes and sufficiently large for the new instar. However the mesh is smaller than the following instar so that flushing the larvae out happens by having the mesh at the lower position and opening the larva outlets. The larvae then flow towards the next water tray and so on to the last water tray, where the mesh holes may match pupae size, to support flushing all or majority of the pupae outside.

In that process of moving larvae between rearing trays, then when the larvae grow and reach their next instar, the mesh is raised and molts are flushed away with the other waste while the live larva remain on the mesh because their size is larger than the mesh. The process then continues between water trays until the last tray which represents the point in time the larva pupate. The outlet from the final larva tray outlets may thus be either L4 larvae or pupae.

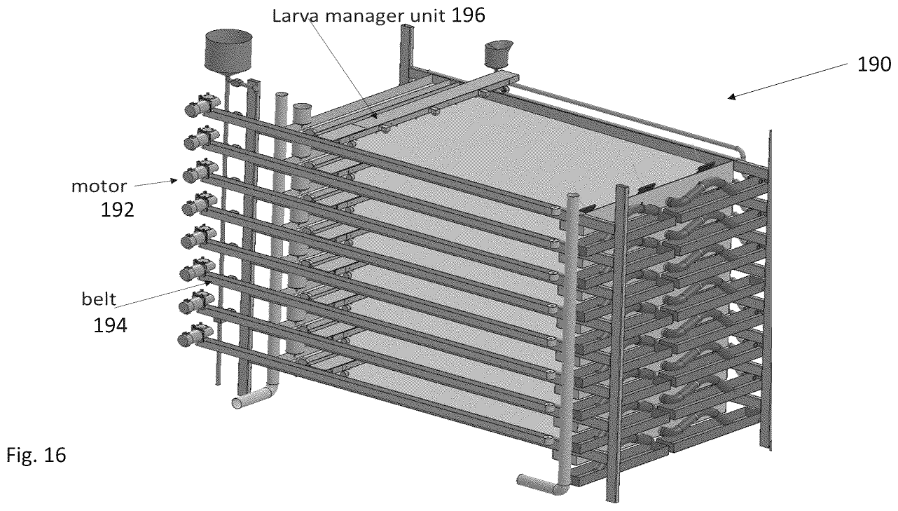

Reference is now made to FIG. 16, which shows an exemplary embodiment of the present invention in which all water tray mobile manager units are connected to a motorized system which may operate each manager unit independently. Thus, each manager unit moves independently of the others in order to maintain, feed, clean and other procedures related to a specific water tray. As shown, the stack of trays 190 comprises multiple conveying systems including a motor 192 and a belt 194 to support the movement of the manager unit 196 along the belt 194 and thus along the respective tray in the stack 190.

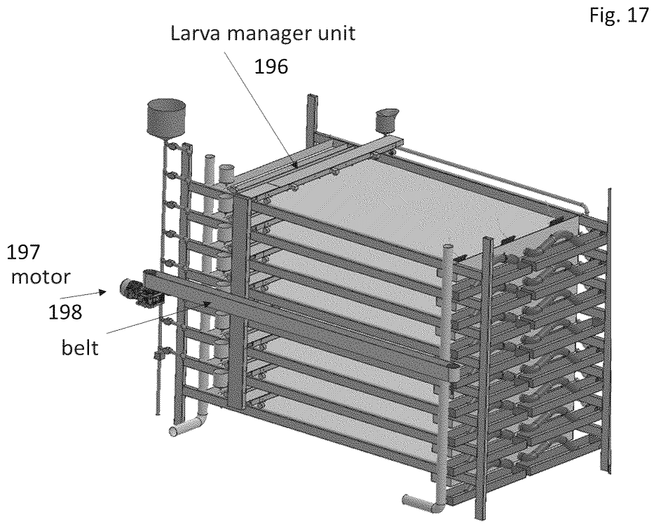

Reference is now made to FIG. 17, which shows an alternative to the embodiment of FIG. 16 in which all manager units 196 are connected together to a single conveying system which move all managers at the same time, together. Thus a single motor 197 and a single belt 198 moves all of the management units 196 in the stack.

In another embodiment the water trays do not include the mesh surface to be lifted and lowered for cleaning. In that embodiment the larva stays in the trays with the same water until flushing out. Automated food dispensing and larva dispensing and distribution is performed by the mobile larva manager unit which travels above the water larva tray.

In an embodiment the mesh surface may include elements extending upwards from the mesh to form cell like shapes. The cells may be closed cells or open, meaning the larvae can swim between the cells walls and move between one cell and another. The mesh may include elements that extend upwards and act like walls or separators or partitions or dividers along part or the entire length of the water tray, perpendicular or in parallel to the tray orientation. Flushing the water with the larvae outside of the tray may involve walls mounted perpendicular to the direction of the water flow, supporting the larvae flowing along the walls and towards the outlets.

In another embodiment, the mesh is fixed in place within the water tray and the height of the water is changed relative to the mesh height. In such an embodiment, the larvae are located above the mesh, and when the water level is above the mesh surface the larva swim and float above the water surface. When and if the water needs to be changed, the water level may be lowered for example by opening outlets at the sides of the water tray, and when the water level drops below the mesh surface, the larva will rest on the mesh surface. Once all the used water is emptied, clean water may enter from the inlet shutter and fill the water tray, as the water level increases and gets above the mesh surface, the larva becomes once again surrounded by water and can swim and float and feed within the water.

In another embodiment the mesh may be rotated around its axis, for example if the water tray is a tube shape. Thus the larvae may alternate between positions above and below the mesh, and may be rotated to be above the mesh when the water needs to be drained. The rotation may be at 90 degrees, or 180 degrees or therebetween to support flushing the larva together with the water when the larvae is required to be flushed outside.

In another embodiment, the water tray can be rotated around its axis. Preferably in this embodiment the water tray shape is of a tube shape. The water tray includes a fixed mesh structure at some height. When changing water is required, the outlet of the tube, located preferably below the mesh level, is opened and the water is emptied. As the water level drops below the mesh surface, which may be measured based on time required to lower the water level or using a sensor to measure the water height, the larvae rest on top of the mesh surface, and then new clean water comes in. Finally the water level is increased relative to the mesh surface allowing the larvae to swim again. When the larvae need to be flushed out, either another outlet above the mesh surface is opened allowing the flow of water with larvae through that outlet, or the entire tube-shaped water tray is rotated around its axis to flush out the larvae with the water. The larvae may be flushed below it to a receiving element which receives or collects the water with the larvae and may convey it to where it is needed. Such a collection element can be another tube, or a water tray or tunnel-shape, or any other form which can support the propagation of the larva and water away from the water tray it was flushed from. The mobile manager unit may be included in such embodiment and function as described above, moving above and adjacent to a single tube shape water tray or above multiple such water trays at once, and dispensing the food and/or larvae above such water trays when needed.

An advantage of using a tube shape like structure is that the upper edges may shade part of the water area due to their curvature, providing a safe environment for the larvae which prefer hiding in dark areas and around edges.

The tube shape may be open from the top in order to allow distribution of food and/or larvae from above.

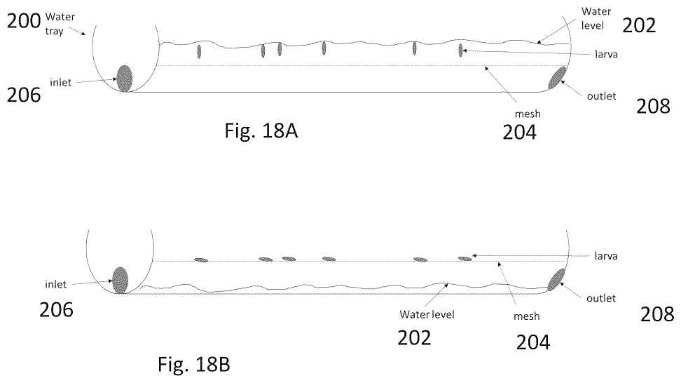

Reference is now made to FIGS. 18A and 18B which are two views of an exemplary embodiment in which the water tray is in the shape of a tube. Tubular water tray 200 is shown in two states. In FIG. 18A the water level 202 is higher relative to the mesh surface 204 allowing the larvae to float and swim inside the water and above the mesh. In FIG. 18B a second state is depicted in which the water level 202 is lower than the mesh surface 204 with the larvae thus resting on the mesh 204. Accordingly, it is possible to change the water by opening the inlet 206 and outlet 208, extracting dirty water and bringing in new clean water. For flushing out the larvae, while in the first state, when the water level is above the mesh, the tube may be around its axis to enable flushing of the water along with the larva.

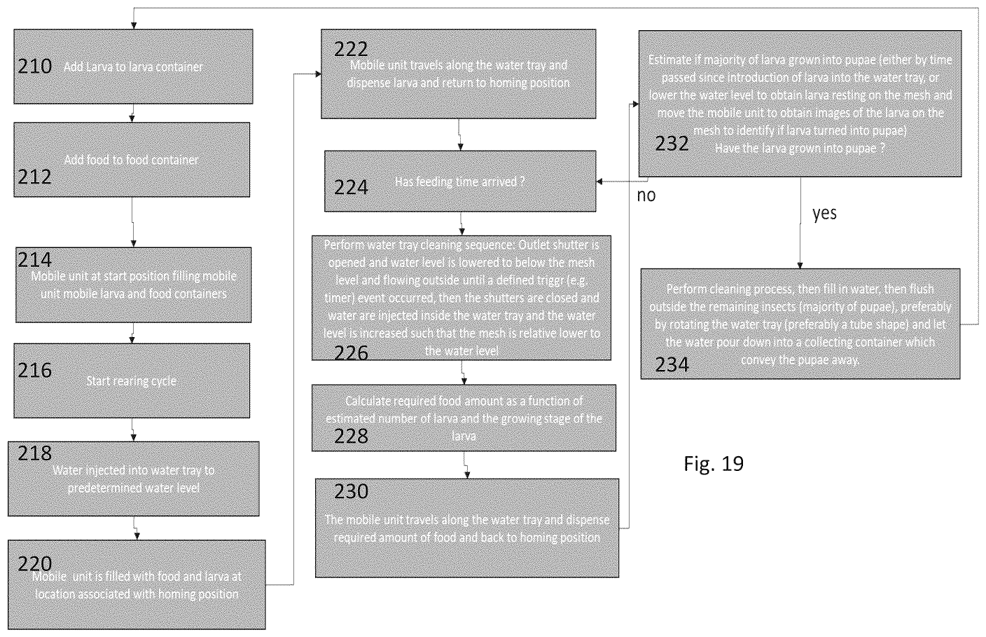

Reference is now made to FIG. 19 which is a simplified flow process describing the general working process when instead of physically changing the mesh level, the water level is changed instead, relative to a stationary mesh.

In box 210, larvae are added to a larva container. In 212 food is added to a food container. In 214 the mobile unit is at the start position and food and larvae are transferred to the mobile unit. In 216 the rearing cycle begins. Water is injected 218 into the tray to a preset level and the mobile unit is at the start position 220. The mobile unit travels along the tray 222 and dispenses larvae evenly, for example using vibrations, and returns to the start position. The system checks for feeding time 224, and if so carries out a cleaning process 226 which involves lowering the water level below the mesh and then changing the water, as described and increasing the water level. In box 228 the amount of food needed is calculated, based on the number of larvae and their stage of development. In box 230 the unit dispenses the calculated amount of food, using vibration etc as necessary for even distribution. An estimate is made in box 232 as to whether the majority of larvae have pupated, and if so the tray is flushed one last time 234 to collect the pupae, and if the tray is tubular then this time the flushing follows rotation of the tube.

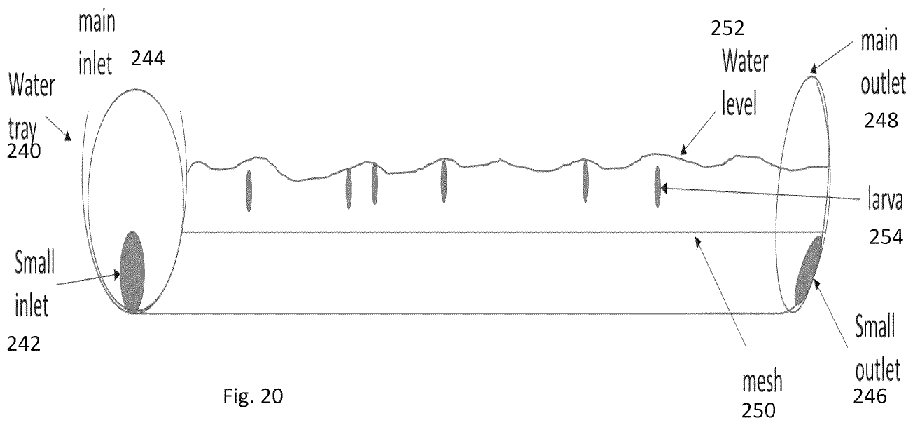

Reference is now made to FIG. 20, which shows a water tray 240 having a rounded shape, and including two different inlets, namely a small inlet 242 and main inlet 244 and two outlets namely a small outlet 246 and the main outlet 248. Mesh 250 is below water level 252 and larvae 254 are swimming in the water above the mesh.

While the main outlet and inlet are closed, a cleaning operation is carried out by opening and later closing the small outlet 246 and small inlet 242 as described above. For flushing out the larvae 254, the main inlet 244 at the first end of the tube and the main outlet 248 at the second end are opened, and water propagates along the entire tube, flushing all the content of the water tray tube shape including the larvae towards the second end, without the need to rotate the tube shape around its axis.

While performing the cleaning operation and changing the water, in all embodiments it is also possible to have water being poured from above, flushing through the mesh and support to flush down through the mesh any additional leftovers (such as small molts and/or food particles) and thus flushing the dirt water outside of the water tray.

An apparatus includes a rack, holding water compartments for rearing larvae, multiple conveyable units, each conveyable unit comprises means for the dispensing of larvae and/or food, and is able to travel above and along a rearing tray water level, hence to support a homogenous distribution of larvae and/or food along the larvae rearing water tray.

The rearing compartments may include mesh smaller than the size of the larva instar or instars that may grow inside the rearing compartment, and the apparatus may have the ability to change the height of the mesh relative to the water height, either by changing the water level or changing the mesh level or orientation (e.g. by rotation)

An apparatus comprising the conveyable unit may include an imaging sensor unit to support identifying the larvae growing stage or determine whether there is a majority of larvae or if a sufficient number of larvae have pupated.

An apparatus includes a rack, holding water for rearing larvae, the rearing compartments include mesh smaller than the size of the larva instar or instars expected to grow inside the rearing compartment, and has the ability to change the height of the mesh relative to the water height, for example either by changing the water level or changing the mesh level or orientation (e.g. by rotation). The apparatus includes multiple conveyable units, and each conveyable unit comprises means for the dispensing of larvae and/or food, and is able to travel above and along a rearing tray water level, hence to support a homogenous distribution of larvae and/or food along the larvae rearing water tray.

Returning now to FIG. 1, and the automated counting of pupa and/or larva is now considered in greater detail, wherein exemplary embodiments cover separation according to sex or other classification features, and transfer into secondary receiving containers with the introduction of another conveyor.

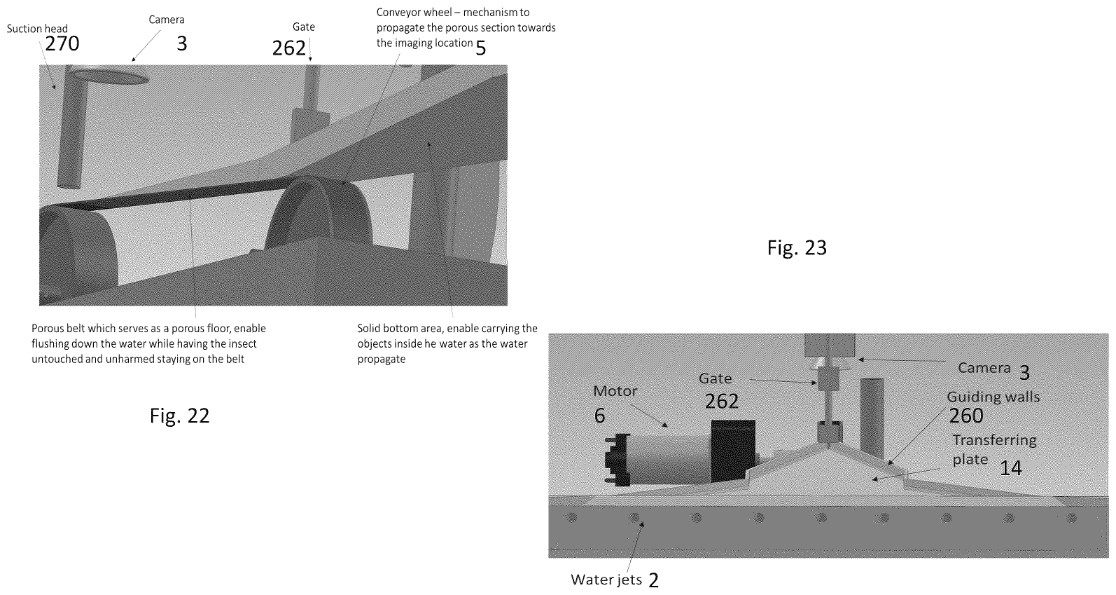

As illustrated in FIG. 1, a transfer apparatus for transferring insects includes water jets 2, a transfer plate 14, camera 3, suction head 270 (see FIG. 22 below), porous conveyor belt 4, conveyor belt wheel 5, conveyor motor 6, receiving container 7, and tilting storage 8.

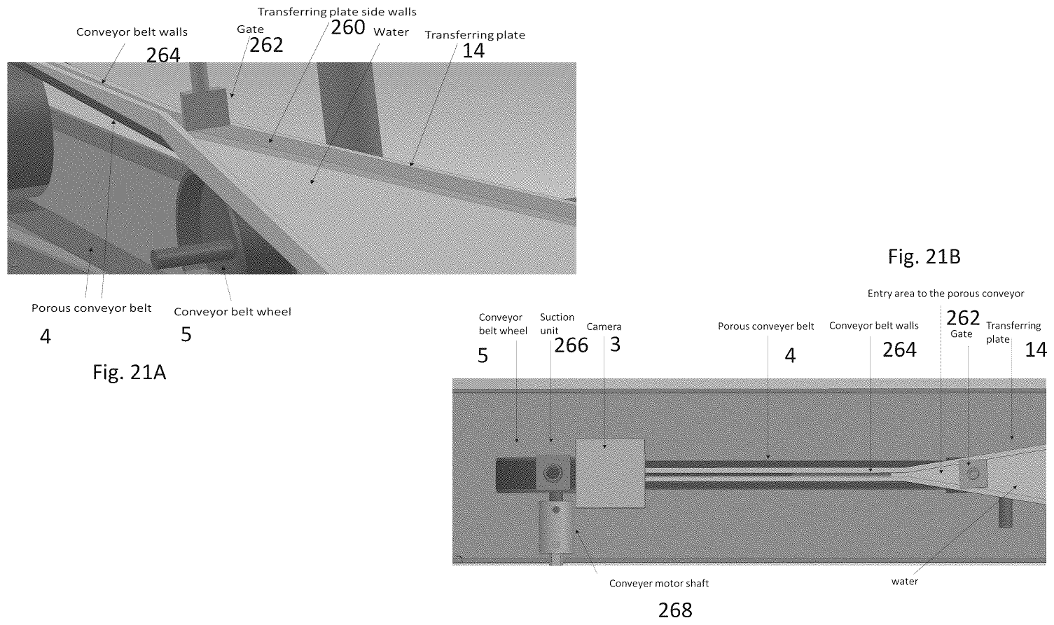

Reference is now made to FIG. 21A, which is a detail of FIG. 1 showing the entrance from the transfer plate to the conveyor. Parts that are the same are given the same reference numerals. The transfer plate 14 has side walls 260 and a gate 262 which leads to conveyor belt 4. The conveyor belt likewise has conveyor belt walls 264.

FIG. 21B is a view from above of the entrance from the transfer plate to the porous conveyor. Parts that are the same as in previous figures are given the same reference numerals and are not described again except as necessary for an understanding of the present embodiment. A suction unit 266 is located downstream of the camera along the path of the conveyor. A conveyor motor shaft 268 may power the wheel 5.

FIG. 22 is a perspective view from the lower side of the area around the camera 3 and the suction unit 266. A suction head 270 extends from the suction unit to pick up mosquitoes that have been identified by the camera 3 as needing to be picked up, as will be described in greater detail below.

FIG. 23 is a view looking downstream of the gate area with the camera 3 in the background.

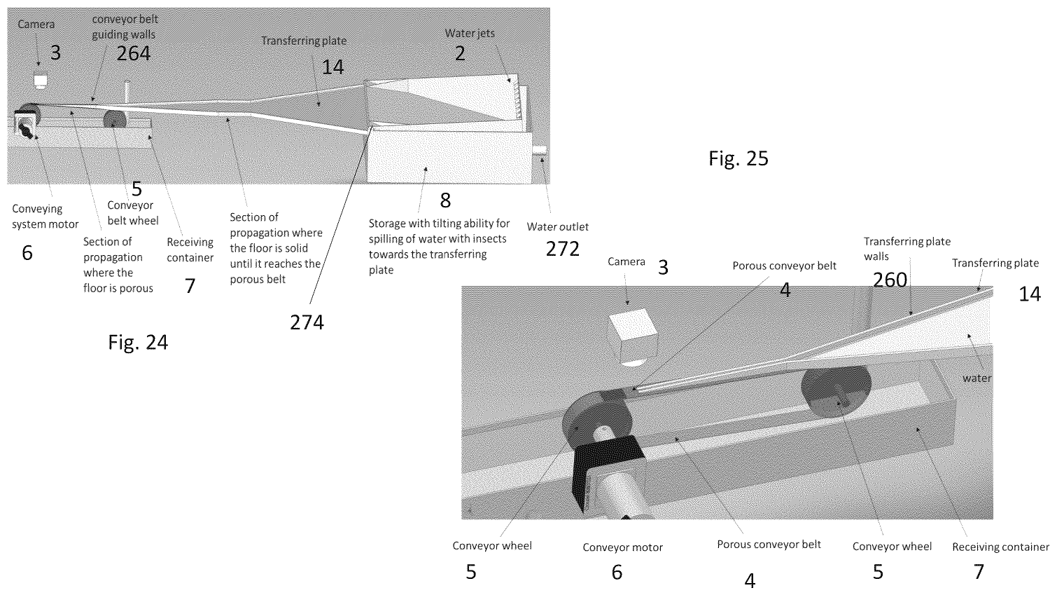

FIG. 24 is a side perspective view of the same gate area. A water outlet 272 allows for water to exit the tilting storage 8. FIG. 25 is an alternative perspective of the gate area.

Aquatic organisms such as pupa and/or larva are stored inside the storage container 8. The storage container 8 may have tilting ability for spilling of water with insects towards the transfer plate 14.

A controller (not shown) commands a tilting mechanism (not shown) to start tilting the container 8 towards the transfer plate 14 to pour the water with insects onto the transfer plate 14. The function of the transfer plate 14 is to transfer the insects towards the conveying mechanism, preferably while guiding and narrowing the flow into a narrow stream to provide a narrow column of water entering the conveying mechanism. When container 8 reaches a certain angle of tilt such that it is now substantially empty, water jets 2 are triggered to remove anything that has got stuck on the container and send that towards the transfer plate 14 as well. The water jets may start working while the container is still in the process of tiling to push the insects away as quickly and efficiently as possible.

Storage container 8 may tilt about hinged connection 274 (see FIG. 24).

The transfer plate 14 may be of any suitable length, and may match the shape of the storage container 8, and may act as a funnel to funnel the insects towards the conveyor 4. The funnel may match with the width of the conveying surface and helps to provide even delivery so that the insects are not bunched together.

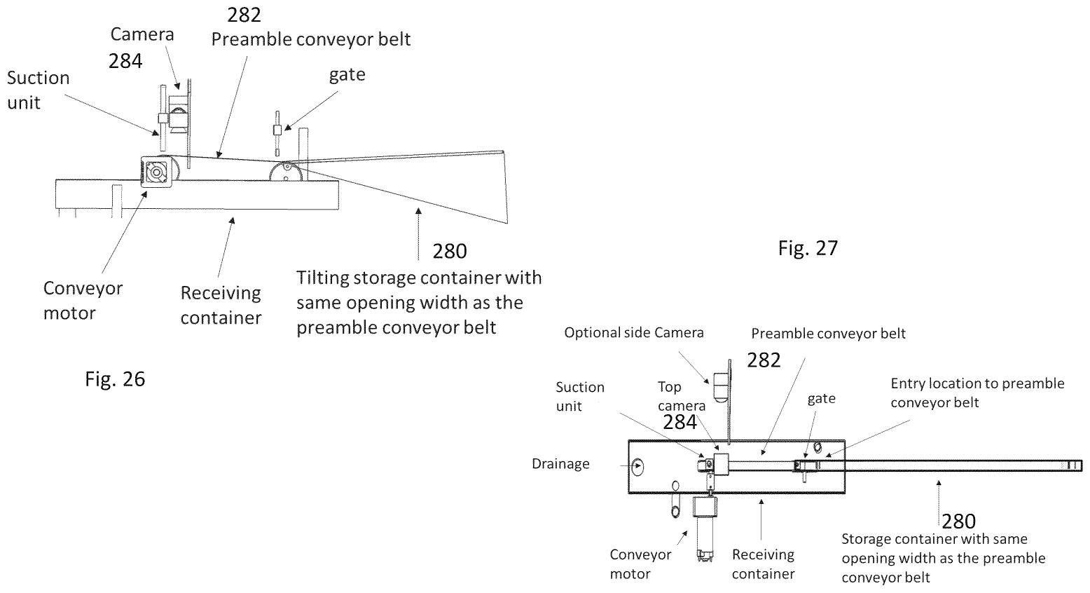

Referring now to FIGS. 26 and 27, it is noted that conveyor and the corresponding camera field of view may be constructed to be the same width as container 8, in which case funneling is not needed and the transfer plate 14 may be dispensed with. FIGS. 26 and 27 are a side view and a view from above respectively of a tilting storage container 280 with the same opening width as conveyor 282, which slowly extract insects to an imaging location adjacent to camera 284.

Returning to FIGS. 1 and 20 to 25, water jets 2 extract anything that remains in the container after tilting and their locations may vary, the jets may be located at the back of the tilting container 8 and/or along the transfer plate 14 and/or adjacent to the area of the entry point into the conveying mechanism. Jets located at the gate area may help separate of insects from each other by firing jets of water at the incoming insects propagating them forward and away from each other.

As mentioned, the belt 4 is porous, for example a mesh, and water drains from the belt as the insects remain on the belt 4.

In an embodiment, the transfer plate may funnel the water and insects into a single line of insects one after the other, thus allowing a single camera from above to view the entire conveyable belt width.

Gate 262 may be provided between the transfer plate 14 and the conveyor 4. Gate 262 may be a mechanical gate that goes up and down, or an electric valve or other means for blocking water flow upon demand. In an embodiment, before moving the water with insects to the belt, the gate is held open when such flow of insects is required, and is subsequently closed when it is required not to send additional water with insects. For example, a classifier at the imaging station and associated with camera 3 may have detected a sufficient number of objects of a required class (e.g. counted a pre-defined number of pupa) and a changeover of receiving storage may be required. The flow of new insects may accordingly be halted until the new storage is ready.

If a gate is present, then the water with insects may flow towards the imaging location, as long as the gate is open.

At the imaging location, camera 3 obtains images of the area on the conveying belt where the insects are located, and if an object is found in the image then the image or images are sent for secondary algorithms to identify if the object is of a group of interest. That is, is the present object a pupa and/or larvae, or is it just dirt. It is possible to use a vision algorithm like YOLO for example in which the algorithm both detects if there are any objects in the image and provides classification results.

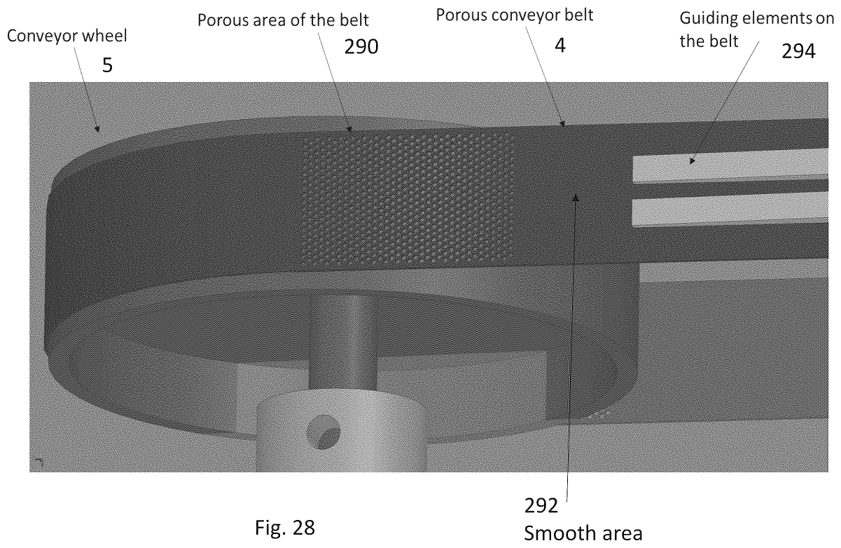

FIG. 28 is a detail of the belt 4 over the wheel 5. As shown there is a porous area 290, a non porous area or areas 292 and guide elements 294. More generally, the entire belt may be permeable or porous, or only part of it may be porous, as shown.

As a further alternative, all of the belt may be smooth and there may be no pores. Water may nevertheless be drained if there is a small gap, say of the order of 0.5 mm and below, between the conveying surface and the guiding elements on the belt, allowing the water to be drained or leak from the sides of the belt but being too small to allow pupae etc to get through. The transfer plate 14 side walls 260 may prevent water spilling sideways before it reaches its required location which is the belt area. That is to say, any gap with the side walls may be restricted to the conveyor belt area.

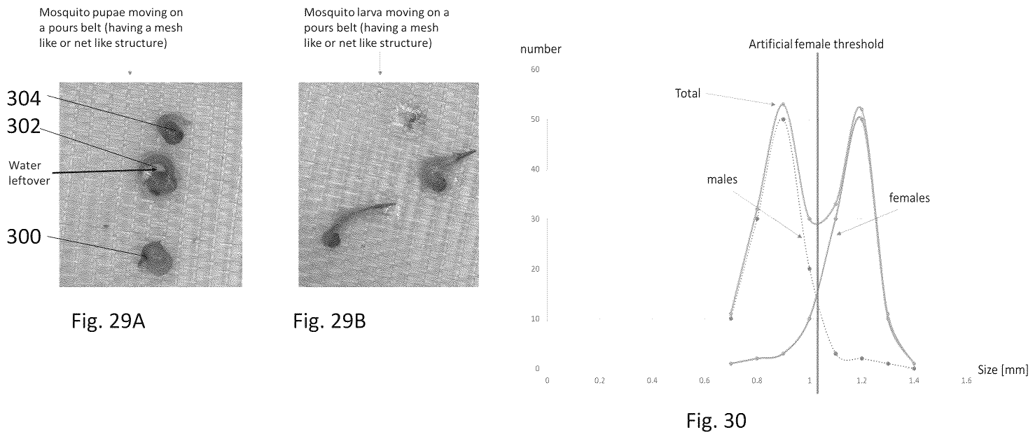

Reference is now made to FIGS. 29A and 29B, which show typical images that may be captured by camera 3 of insects in pupae and larvae forms respectively, passing below on belt 4.

The images may be used to count the number of insects. Counting requires determining which items in the image are in fact insects and keeping a tally. For counting, after obtaining the image, the conveyor continues on its way, following wheel 5 around and submerging in water on its return path. As the belt is under the water, larvae can freely swim away from the belt and pupae may be expected to fall off. Alternatively, insects can be flushed away using water jets after being imaged and identified. Alternatively the insects may be classified into groups that are wanted and not wanted or that are wanted in different places in which case the classification may be followed by separation or sorting as will be explained below.

If only counting is required then following counting the larvae pupae may be left in water container 7.

In a further embodiment, insects may also be poured directly onto the porous conveyor belt 4 from the storage area without using a transfer plate, for example as shown in FIGS. 26 and 27. In such an operation pouring is relatively slow, to ensure sufficient separation between the insects, and preventing piling of the insects onto the conveyor belt.

In embodiments where insects are flushed away from the belt using water jets or by suction or blower, the belt 4 may not necessarily submerge on the return leg.

One reason for draining of the water is to provide a clear view for imaging. Thus drainage may not need to be complete but merely sufficient to allow for undistorted imaging. An alternative to using a belt is to use a rotational plate used to convey the insects.

Accordingly, utilizing a conveyor or other conveying surface to convey aquatic insects while water is drained away, may allow for clear imaging without distortion by the water and may separate insects from each other as they move along the conveyor so they can be identified, classified and sorted individually as needed.

Letting the water drain also lowers the volume of water per surface area resulting in lowering the velocity of the insects and better controlling the velocity of propagation of the insects, which results in better images obtained at the imaging location leading to easier identification and manipulation of the insects.

In an embodiment, mosquito pupa sex (male or female) of the mosquito pupa located on the permeable conveyor belt is identified at the imaging station based on the pupa size.

There are mosquito species (e.g. Aedes albopictus, Aedes aegypti) which have different sizes of male and female pupae. Under good rearing conditions (e.g. providing sufficient swimming area for the larva in the water), typically the female pupa would be larger than the male pupa.

In an embodiment, as the pupae are carried on the porous conveying system (porous conveyor belt) the pupae are imaged at the imaging location and their sex is then identified based on their size (e.g. the area they occupy on the belt) and the computer software classifies as male or female accordingly. Thus in FIG. 29A, pupae 300 and 302 are relatively large and are classified as female. Pupa 304 is relatively small and is classified as male.

In an embodiment, the imaging may be calibrated so that any detected pupae is defined as male if its size is lower than a specific threshold and identified as a female if its area is larger than a specific threshold. These two thresholds may be the same threshold value.

In an embodiment, the imaging and classification system may calibrate itself, say based on examples, to learn the estimated size of the typical male and the typical size of the female pupae.

Based on size only, in the image above, it is assumed the upper most pupa is male while the other two are females.

Yet, it can also be that those three are females, with the two largest once being large females, and the third simply being a small female, and males are then typically smaller than the smallest pupa in this image.

And so additional embodiment may apply another method to decide if the size correspond to a male or a female:

The imaging and classification system may accordingly extract from the obtained images the sizes of a first batch of individual objects.

During the automated calibration process, the system may not classify them as either category (of pupa male or pupa female). After recording the size of multiple number of pupa objects, the system may extract typical sizing for small and for the large objects, for example by finding two means around which the results cluster.

The graph shown in FIG. 30 illustrates a further embodiment in which sizes are shown against numbers of individuals. Two distinct peaks are present with a trough in between. The two maxima points may be identified and then an artificial threshold is selected in the trough therebetween.

Using the above method, the threshold may be changed dynamically as additional pupa may arrive that have undergone growth under different conditions. Thus the threshold may remain accurate despite changes in the incoming population.

In the second graph it is demonstrated how with different rearing conditions if the gap in size between the typical female and male is smaller, then the threshold is different.

The expert can decide how many objects the system records before setting the threshold, be it dozens or hundreds for example.

The dimension on the X axis may correspond to the thickness of the pupae which has direct linkage to the size of the pupae. The Y axis may represent a very general example of a few hundred pupae with different sizes, as the Y axis is the number of pupae per each size on the X axis.

If the rearing conditions are not ideal, the difference between the male and the female pupa size may be less obvious.

One may alternatively apply a simple average size of grown males and females in order to make the decision simpler, yet probably less accurate.

Locating objects on the image may be implemented by using classical vision algorithms or can be implemented by deep learning neural network algorithms which can also be trained to classify such objects are mosquito pupa, and may be trained to classify additional classes such as mosquito larva.

The classifier may then classify male pupae and female pupae based on their size, with males having a size smaller than a male threshold and females having a size larger than a female threshold. The male and female thresholds may be of the same value or different values, and their values may be arbitrary or calibrated, or estimated, as discussed.

The pupae sex (male/female) which are imaged at the imaging location after being separated from the water and conveyed using the permeable (or partially permeable) conveyor belt, according to the different detailed options above, may be classified using size as described, but, alternatively or additionally classification may be made to involve identifying visual features on the body which positively identify if the pupa is a male or female. These may reinforce the size based results or may be instead of size based results, and different combinations may be appropriate for different species and different growing conditions.

The classification is preferably done using a convolution neural network (CNN) which has been trained to identify such features, and distinguish between classes of males, females and/or unidentified (or “other”).

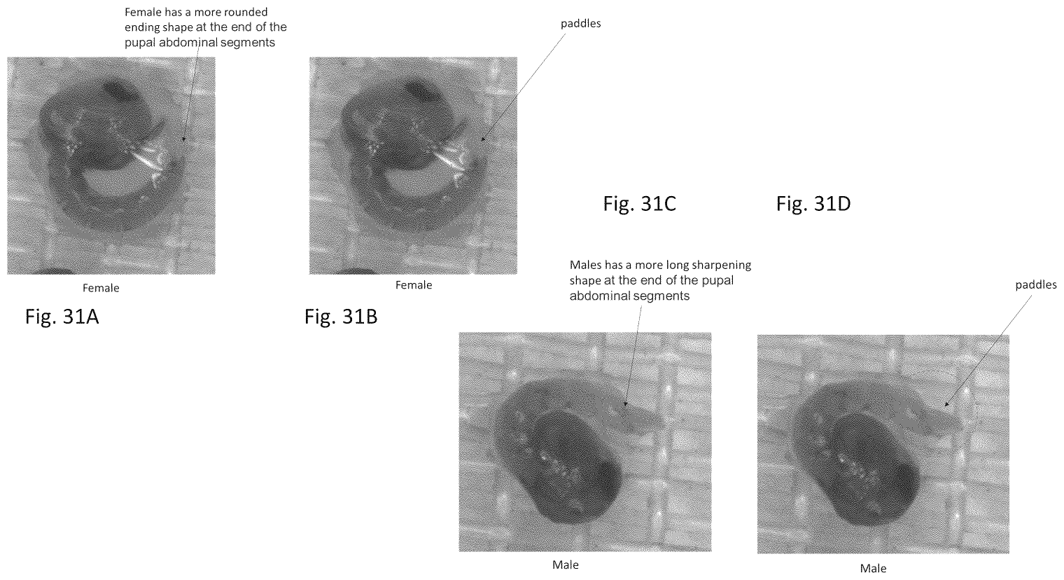

Such an electronic classifier may for example be trained to identify the sex based on the shape of the end of the abdomen section corresponding to male or female as shown in FIGS. 31A to 31D. As shown, the male has a more tapered end of the abdomen than the female.

Such an approach ensures there is no bias due to size differences, having for example a small female or a large male.

Following classification, an operation may be carried out on the pupae, such as counting and/or transfer the pupae into a water container, and/or extracting the classified insects based on the result of the classification, for example to perform sex separation of mosquito pupae.

An embodiment may use an image to identify the end of the abdominal segment, and may then identify a male from a long tapering shape at the end of the pupa abdominal segments; and may identify a female from a more rounded tapering shape at the end of the pupa abdominal segments.

The classifier may only be trained to identify a pupa female body part as described above and an extraction operation may be carried out only on identified females. Alternatively the classifier may identify only the males and extraction may be of the identified males.

In another embodiment, a permeable conveyable surface for imaging of insects is used to support separation of larva based on their fluorescence excitation to specific wavelengths.

At the imaging location, imaging carried out using a light at a wavelength that causes objects that were engineered for that purpose to emit florescence light. The emitting light and excitation light may be of different wavelengths to make the analysis easier. The insects may be engineered so that specific body parts fluoresce, e.g. red eyes, green testis etc.

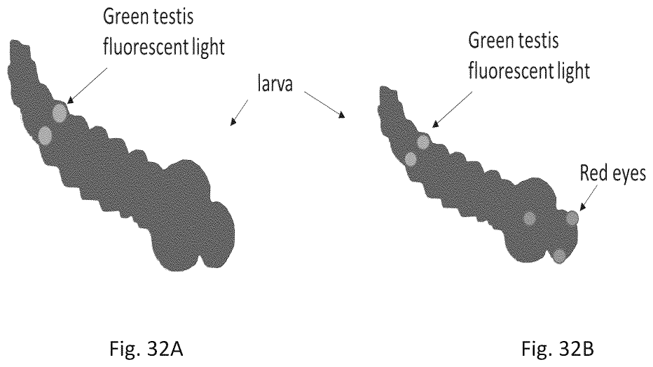

FIG. 32A shows a male mosquito larvae with green fluorescent testis and FIG. 32B shows a mosquito larva with both green testis, red eyes and red dot around the head.

Accordingly, the storage container may contain larva which are emitting fluorescence light upon excitation.

Unsorted larva may thus be engineered with individual body parts with various fluorescent markings and held in a water container.

The larvae may be taken to the imaging location as described in any of the embodiments above, and images may be obtained using regular visible light.

A first electronic classifier may identify larva objects in the obtained images and identify location and classify specific body parts e.g. larva head, larva abdomen etc. in the image.

The regular light may now optionally be shut off.

Light of the excitation wavelength is then switched on and a second electronic classifier identifies presence of emitted fluorescent light and its location in the image;

Correlating may be carried out of the location of the fluorescent emitted light with the body part previously identified.

The larvae may then be classified according to the fluorescent light emitted. Thus X number of larva may be counted and classified as having red eyes and Y number of larva may be classified and counted as having both red eyes and green testis.

As before, operations are carried out on such larva according to the classification which may include counting and/or the transfer of the larva, thus for example transfer of the red eyed larvae into a first vial, for example using a suction head, and transferring larvae having green testis preferably using a suction head, into a second via.

A robotic arm may operate the suction head or any other transfer means used.

In another embodiment, identification of the objects under regular light is carried out, but identification of body parts is dispensed with, and classification is based on the wavelength of fluorescent light that corresponds with object location.

A further embodiment may dispense with detecting the objects at all and go straight to detecting fluorescent light.

The use of an imaging sensor such as a camera, alongside the fact that the object is not moving or is barely moving since it is not in water, ensures more accurate imaging.

Using the first of the fluorescent imaging embodiments above, it is possible to automatically classify a specific body part e.g. head, eye, abdomen etc., and identify if the body part is being excited at a specific wavelength. By contrast when using flow cytometry, a laser may cause the necessary excitation without feedback as to which body part caused the excitation. The present embodiments may allow multiple different fluorescence marking per insect, on different body parts, and may classify each insect according to several independently detected features, thus strengthening the probability of a correct determination.

The present embodiments may allow for sorting according to insect type (e.g. pupa or larva) or insect sex and/or type (e.g. larvae or pupa male or pupae female), and also sorting according to which body part fluoresces and at what wavelength.

In a further embodiment, a first electronic classifier may identify larva objects in the image.

A second electronic classifier may then identify presence of emitted fluorescence light. The fluorescent emitted light is then correlated with larva detections of the first classifier. Accordingly each larva is classified according to the detected excitation color, resulting in counting different groups, e.g. X number of larva counted and classified as having red marking and Y number of larva classified and counted as having green marking.

Operations may then be carried out as before, for example to transfer larvae emitting red into a first vial preferably using a robotic arm and transferring larva emitting green color preferably using a robotic arm into a second vial.

In another embodiment, only counting of the objects is required without classifying them.

In such a case instead of a camera, any kind of sensor for detecting objects on the permeable conveyor belt may be sufficient. Examples of sensors that may be used are a laser emitter and detector or an optical reader mounted on the conveyor belt from the side, and which is triggered each time an object passes.

In another embodiment, the separation surface is fixed in position.

Insects are propelled toward a permeable surface from which the water is drained. Arrival of the insects at the permeable surface is controlled to receive individual insects one at a time, say using a shutter or gate onto the permeable surface.

One or more images are taken at the permeable surface, and classification and any sorting or other operations are as for any of the previous embodiments.

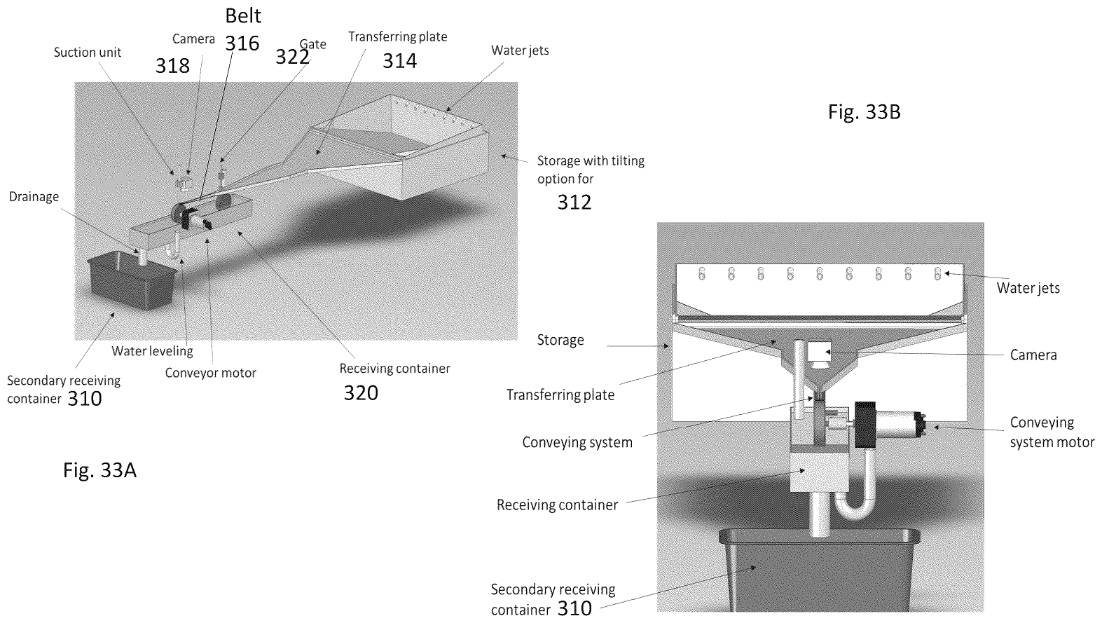

Reference is now made to FIGS. 33A and 33B which show secondary receiving containers 310. The transfer system works as before for transferring insects from a source container 312 via a transfer plate 314 to a belt 316 and a via a camera 318 for collection in a primary receiving container 320. Once the number of pupae and/or larva etc has reached a certain threshold, the system stops sending insects to the conveying system, for example by stopping the conveyor, and/or stopping pouring water with insects towards the conveying mechanism and/or closing gate 322.

The receiving container is then being drained into secondary receiving container 310, which may in turn be exchangeable when full and may be one of many secondary receiving containers. Hence once the secondary container is full, it may be removed and replaced as needed.

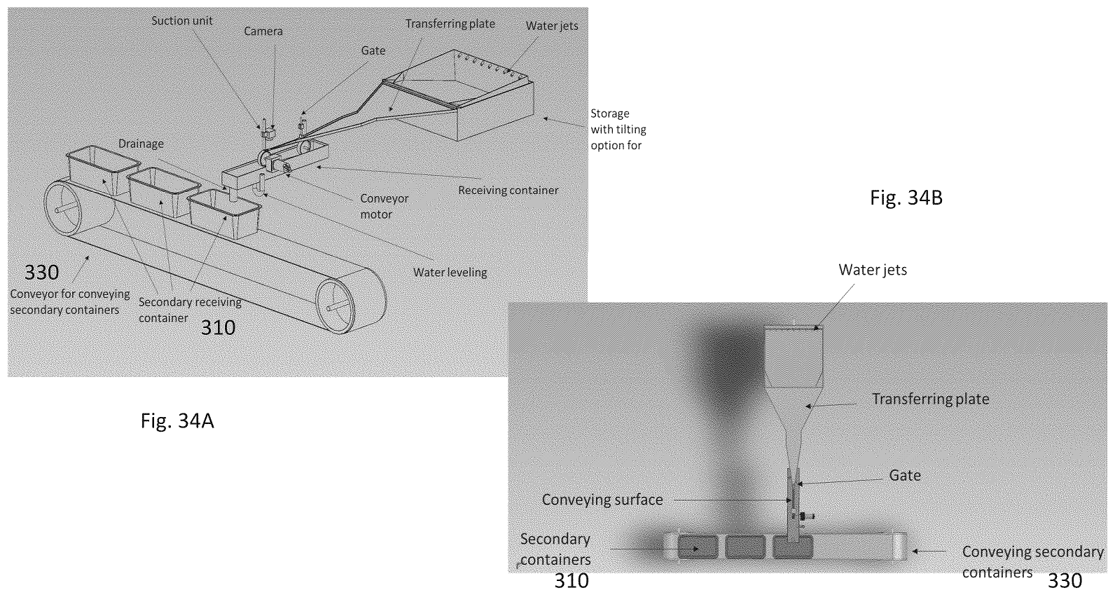

Replacing the filled secondary receiving container with an empty secondary receiving container is preferably done automatically utilizing a conveyor moving emptying secondary receiving containers and placing a new empty secondary receiving container to receive insects from the (main) receiving container. FIGS. 34A and 34B show a conveyor 330 for exchanging the secondary containers 310.

Reference is now made to FIG. 36, which shows a variation in which transfer plate is dispensed with. Instead, storage container 332 tips directly onto conveyor 334 and guides 336 carry out the funneling to center the stream at the lower end.

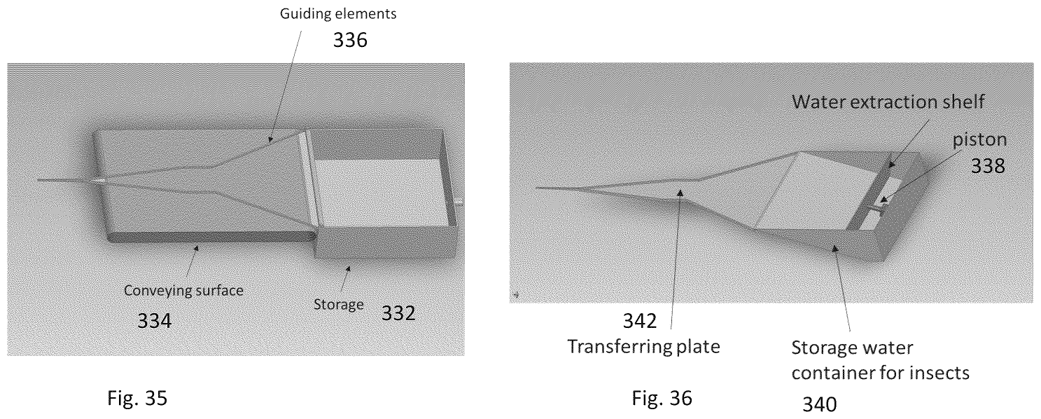

Referring now to FIG. 36, a piston 338 pushes the insects and water from storage container 340 onto the transfer plate 342. It will be appreciated that the present embodiment may be combined with that of FIG. 35 to dispense with the transfer plate as well.

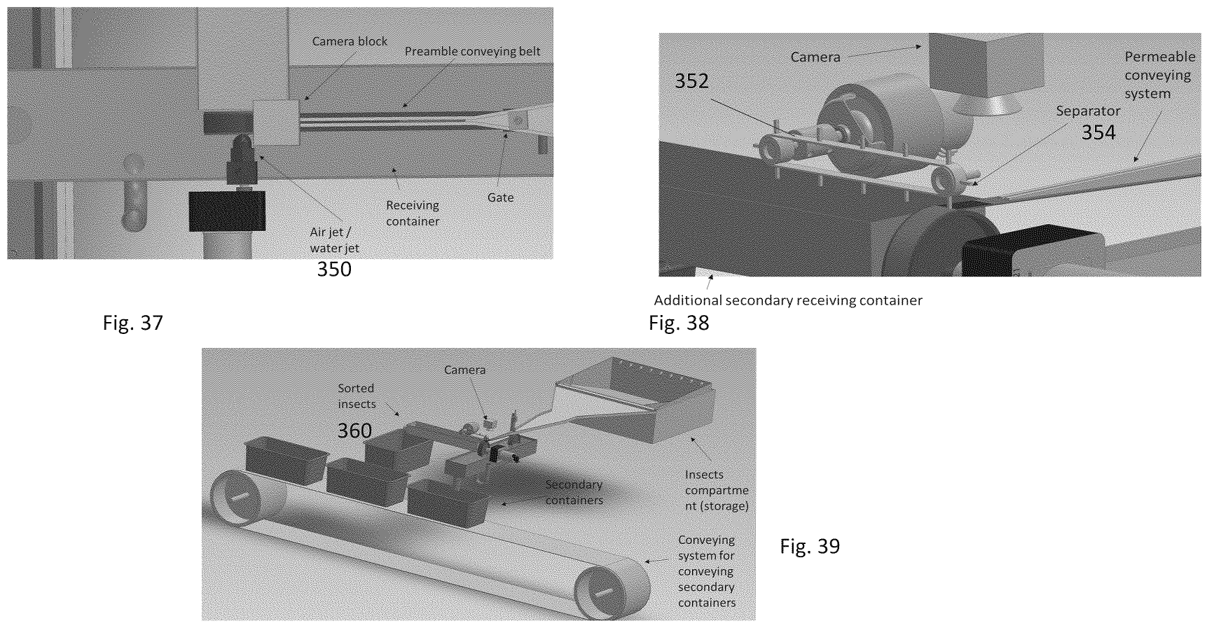

FIG. 37 shows an embodiment in which sorting is carried out by using a jet of water to remove the insect of the desired classification. The insect is extracted by using nozzle 350 which ejects water or water jets and pushes the insects off the belt as required.

Referring now to FIG. 38, instead of using an air jet or water jet to extract and move the insects from the imaging location, a separation conveyor 352 with picking elements 354 may be provided. An insect is classified as wanted and the next picking element 354 on the conveyor picks the insect resting at the imaging location and places it on the separation conveyor 352.

Reference is now made to FIG. 39 which illustrates a system for collecting two classes of insect. Insects are imaged on the conveyor as before, collected into primary and then secondary containers as before. The secondary containers are changed as described above with respect to FIGS. 34A and 34B. to provide continuous separation and filling of secondary containers with counted insects of the same classification type. In addition, another container 360 collects insects classified into a second class.

The above provides the ability to have a continuous input of relative non-moving aquatic insects towards an imaging location, to be analyzed and then sorted as required. Thus, as required, the insects may be sent to the receiving container, or may be picked by a suction head to sort between two categories. The suction head may extract different categories into different receiving containers based on visual features, for example all red-eye insects being placed in container 1, all green abdomen pupa being placed in receiving container number 2, etc.

Relevant to all embodiments herein, the identification camera may be placed atop of the conveyor belt, and/or to the sides and/or also below the belt, in order to provide also a bottom view of the specimen under investigation.

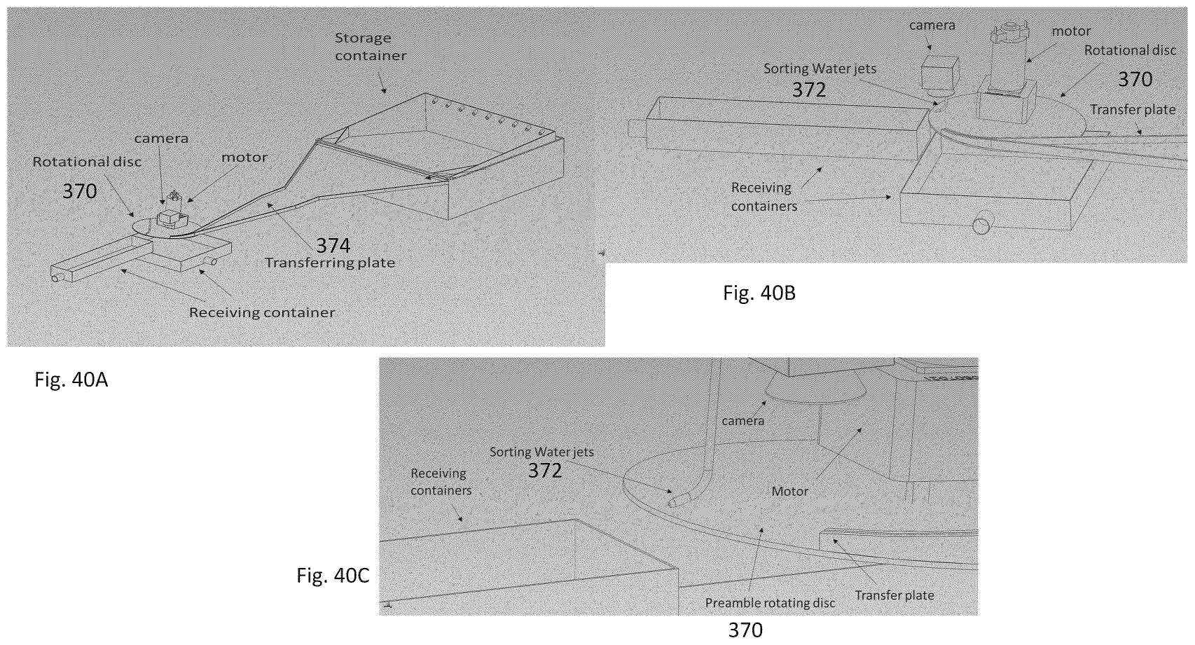

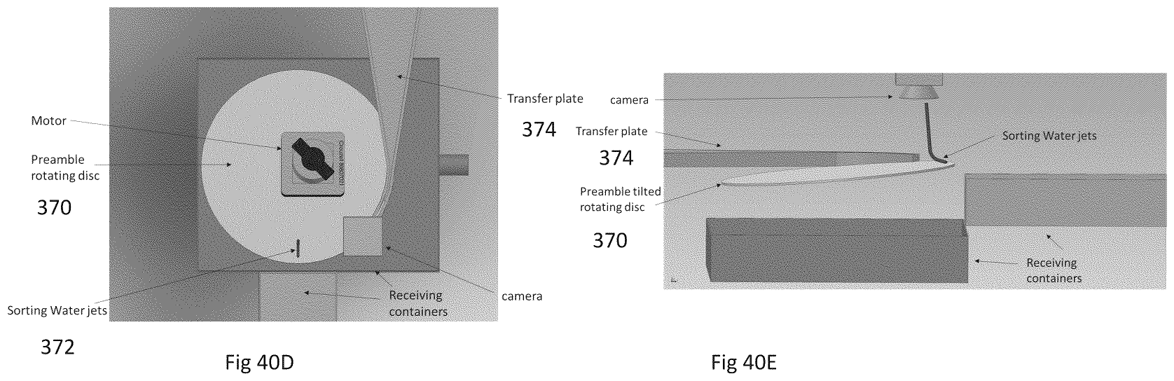

Reference is now made to FIGS. 40A, to 40E, in which the conveying system is made of a permeable rotational disc 370 rather than a conveyor belt.

The rotational disc 370 may be mounted at an angle thereby to support extraction of the insects from the disc.

The disc is permeable or partially permeable.

As disc 370 rotates, it conveys and bring the insects towards the imaging station.

Along the disc 370 there may be mounted multiple extraction mechanisms such as multiple water jets 372 to sort the insect towards different locations according to the electronic classifier results.

In the present embodiment a transfer plate 374 or any other embodiment herein may be used to provide a flow of insects towards the permeable rotational disc 370. The insects may be as any of the embodiments above. Multiple water jets around the disc are possible each associated with different receiving containers, thereby to sort into multiple different classes.

FIG. 40E is a side view showing disc 370 mounted at an angle. The angle may be controlled using an actuator in order to tilt the disc each time towards the receiving container.

The terms “comprises”, “comprising”, “includes”, “including”, “having” and their conjugates mean “including but not limited to”. The term “pupae” may also refer in this document to individual pupa depending on the context. The term “permeable” refer to materials which allows liquid to flow, leak or flush through it, such as permeable or porous surfaces.

In another embodiments, instead of using in the embodiments above a permeable surface, then a spongy or an absorbent material is used which can absorb the liquid and provide the functionality of the permeable or partially permeable surface as described above which is the separation of water from the insects that remain on the surface (e.g. insects remaining on the spongy or absorbent surface).

Returning now to FIG. 2 and the racks are considered in greater detail.

Typically, mosquitoes are reared in racks, holding multiple trays in each. An egg hatches in water, becomes a larva going through four instars, L1 . . . L4 as explained above and a few days later, it becomes a pupa. Subsequently the adult mosquito emerges from the pupae.

Typical racks hold hundreds of thousands (each tray can typically hold 5K-20K depending on its internal surface area) of larva, with some 30-50 trays one on top of another. When the larvae pupate, in the known art, technicians then empty all the trays at once into a pool.

This then causes them to manually group pupa into batches for continuous rearing inside cages—the pupae turn into adults and they must either be inside a cage, or a release tube if they are to be released as part of a sterile mosquito campaign. They cannot be allowed to fly freely in the lab.

As a cage can only contain a certain number of mosquitoes, between a few hundred and a few thousand, the pupae from the rack may be grouped/counted many times.

Another problem is that not all larva in all the trays pupate at the same time, and you may spill larva which are still larva into the big pool together with the pupae.

Another challenge is the requirement to feed the larva every day, and potentially even multiple times a day. When scaling the system for millions and tens of millions of insects this requires a lot of man hours.

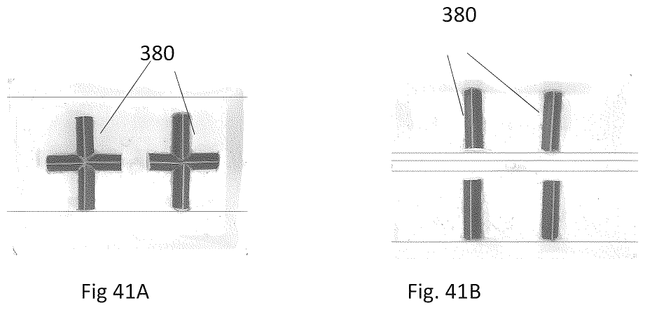

Referring now to FIGS. 41A and 41B, larva prefer hiding places when they sense danger. The water trays may include internal geometry as shown by structures 380 to increase the areas where the larva can rest against to.

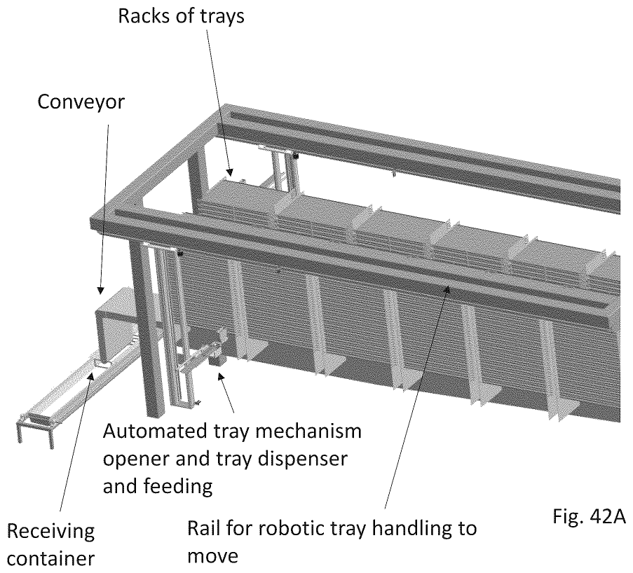

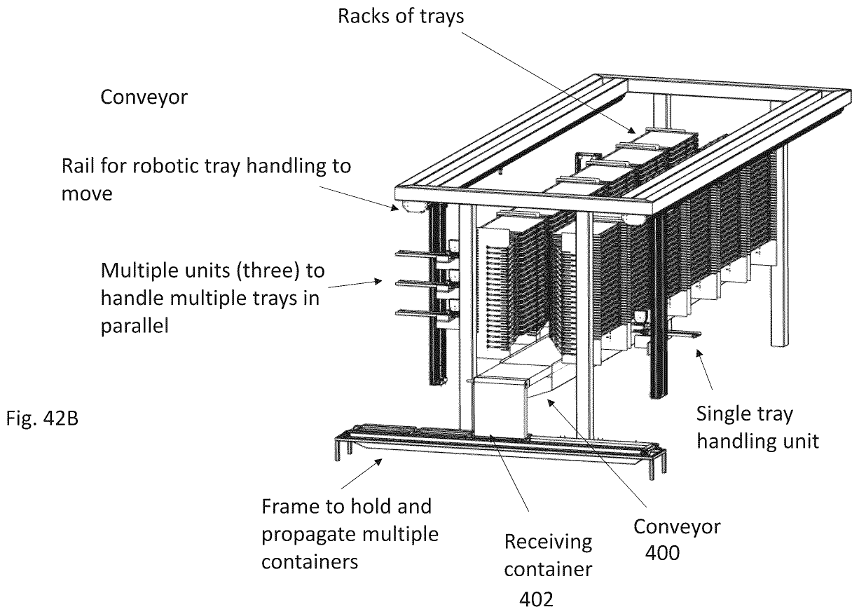

Reference is now made to FIGS. 42A and 42B which are modifications of the rack of FIG. 2 to [1] feed the larvae automatically while moving between trays, [2] how to extract each tray, and [3] how to collect and move the larva into a container.

As shown in FIGS. 42A and 42B, trays containing water and larvae are placed in racks, the racks enable pulling out of each tray independently and pushing each tray to the end of the rack in order to pour out its contents. A robotic element may be provided to pull or push the tray on the rack.

A rail or other means allows travel of the robotic system along the racks as discussed hereinabove, and the robotic system is able to position itself accurately in front of the tray it is now working on. Accurate positioning may be at the level of mm. A feeder is associated with the robotic system and may dispense food when pulling the tray to its open position. A conveyor 400 may receive water poured from the tray and convey the insects towards a receiving container 402. The container may receive an estimated number of insects from a single tray

To pour out the content the trays may be pushed or tilted in different ways, and more than one robot unit may operate on the rack in parallel in order to increase productivity.

Conveying the insects towards a receiving container may involve a porous conveyor, so water is flushed but insects remain. Alternatively, the tray may be emptied into a pool with a slope or other means to support propagation of the water towards the receiving container.

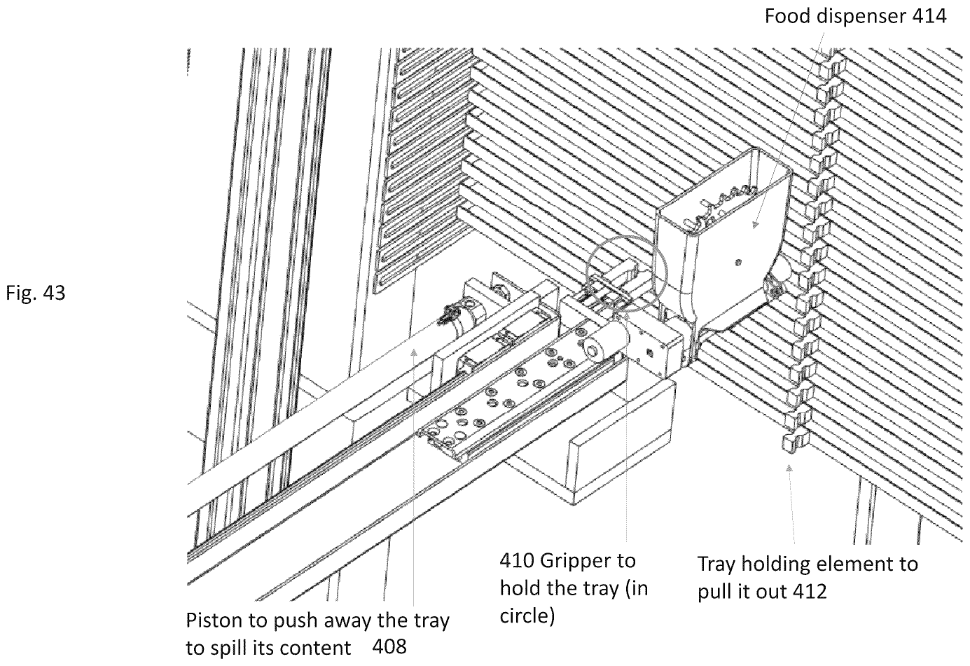

Referring now to FIG. 43, the trays are each opened by pulling out to the edge of the rack by an automated or robotic system, for example using a piston 408 connected with a gripper 410. The gripper may grip the tray at a gripping position and pull it out. The tray may have a holding element 412. Once pulled out, the food dispenser 414 may access the tray

Once pulled out, a feeding mechanism may drop food into the tray. Other treatments can be added, such as adding larva or water when the tray is open. Also the robotic device pulling out the tray may dispense the food at multiple locations above the water surface, and may also have an additional sensor or sensors to measure larva rearing parameters. For example a camera may take an image from above of the water surface inside the tray, to estimate the total number of live insects in the water in order to calculate a required amount of food to dispense, or to identify a number of pupa in the water to better decide at what time to flush the water tray, or to identify the starting time of pupation to better predict the flushing time.

When a few days later the larva becomes pupa, and it is necessary to extract the pupa and remaining larva, then each larva tray is pushed forward and placed at an angle to pour its contents to a receiving element.

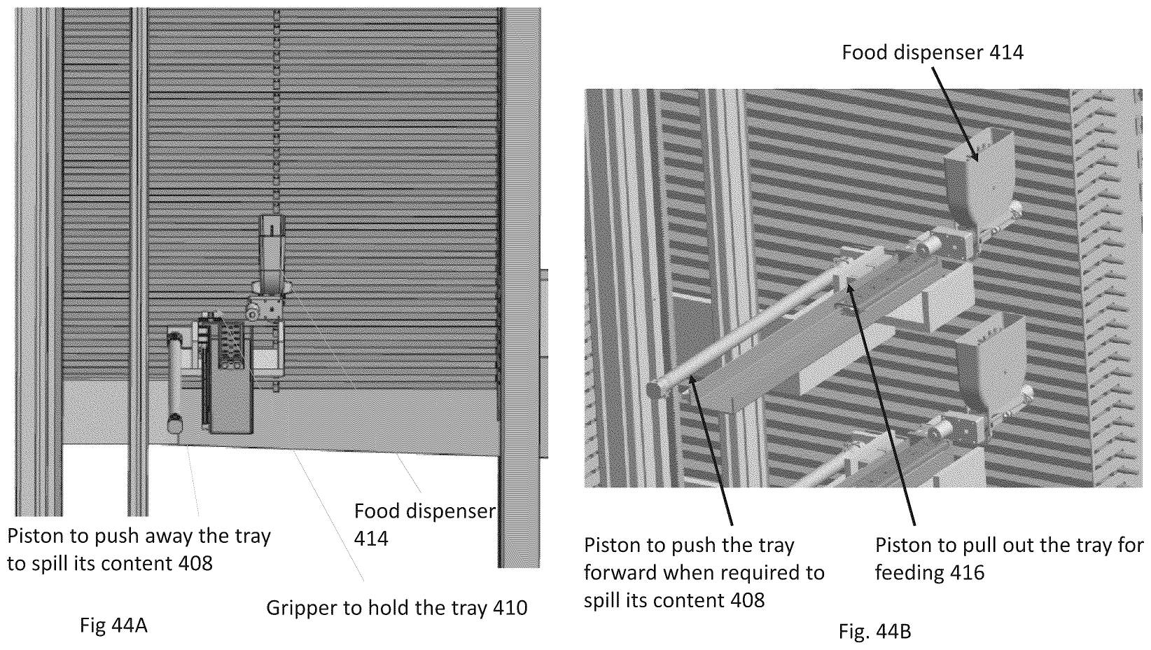

Referring to FIGS. 44A and 44B, the food dispenser 414 may have a container with food, a dispensing opening to allow food to be dispensed, and an auger or a spiral dispenser to support measured dispensing of food at each dispensing event. There are many standard units for dispensing of solid food that can be used on the device and these are just a few examples. Two food dispensers are shown associated with a single rack as well as a piston 408 to push the tray forward to pour out the content and piston 416 for pulling out the tray for feeding.

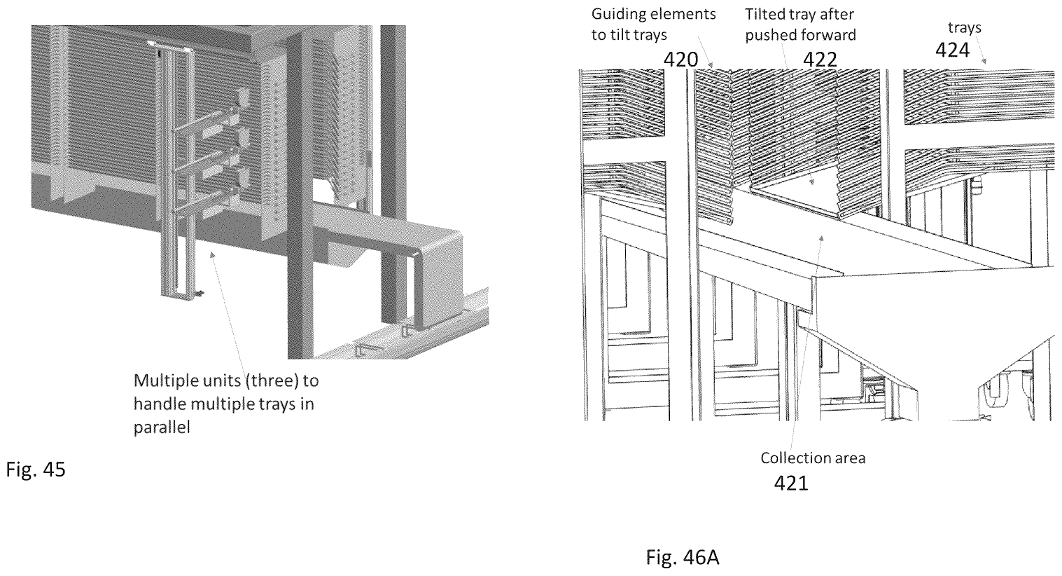

Reference is now made to FIG. 45, which shows the rack as before but this time with three units with gripper, feeding unit and pistons, to handle multiple trays in parallel. It will be appreciated that three units are purely exemplary and any suitable number that fits the rack may be used.

Using the present embodiments, all trays may be fed every day, and, as discussed, potentially multiple times a day.

When required, the next step is to flush each tray separately, and collection of the poured water may be into a collection basin or onto a conveyor, which may be porous as discussed hereinabove.

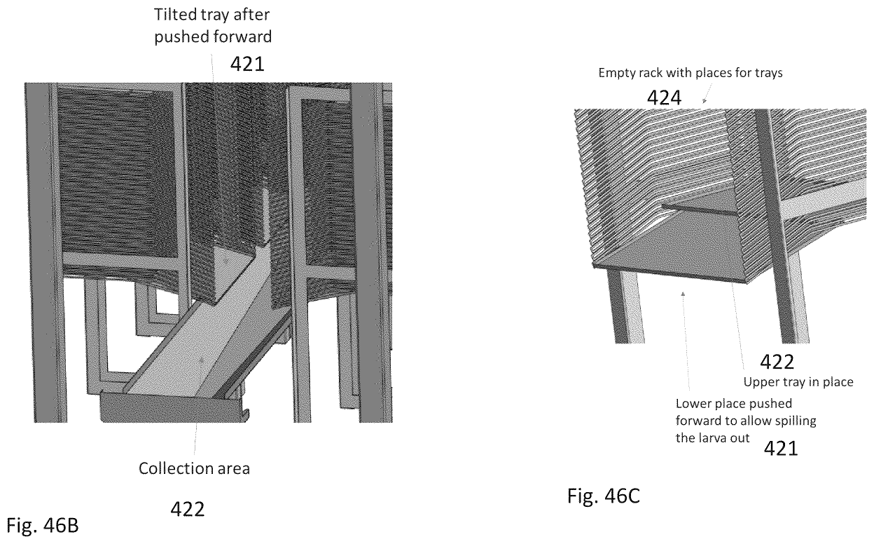

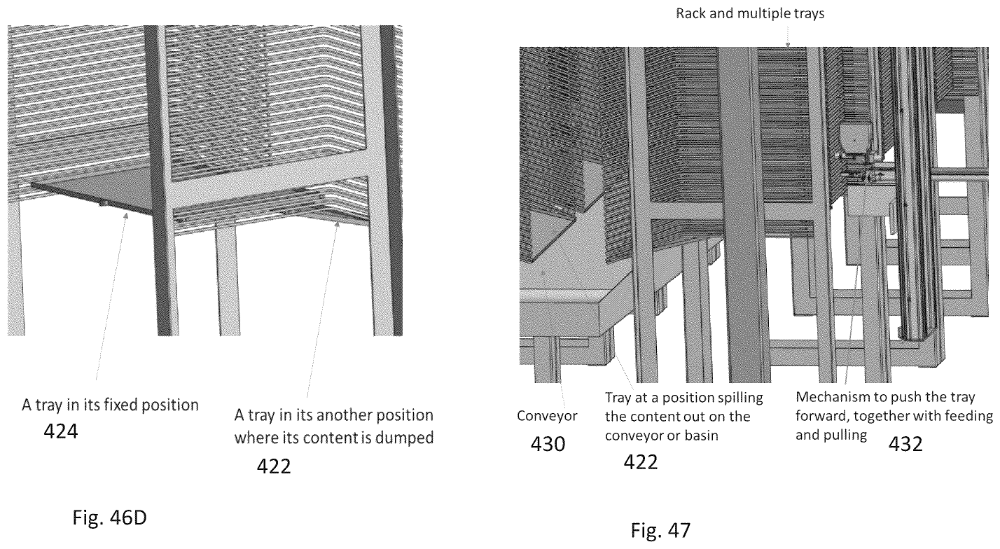

Reference is now made to FIGS. 46A-46D which show trays in various positions in and out of the rack. Guiding elements 420 guide the trays into specific tilts for pouring into a collection area 421. Tray 422 is extended into the guides for pouring. Tray 422 remains in place in the rack 424.

As discussed, using the two pistons or using any other method, the rack has the ability to both pull an independent tray in order to feed it, and may allow pushing the tray to the other side to pour the water.

FIG. 47 shows a conveyor 430 and shows trays 422 pushed to a pouring position to pour into the conveyor 430. A second mechanism 432 pushes the tray in the other direction for feeding. Upon pouring the tray content, the larva may alternatively be poured directly into containers which are waiting to be filled. Such containers may be positioned on the moving conveyor which then brings the next empty container to be filled when required. Each container may be filled with an estimated amount of pupae coming from an exact number of trays as discussed in earlier embodiments.

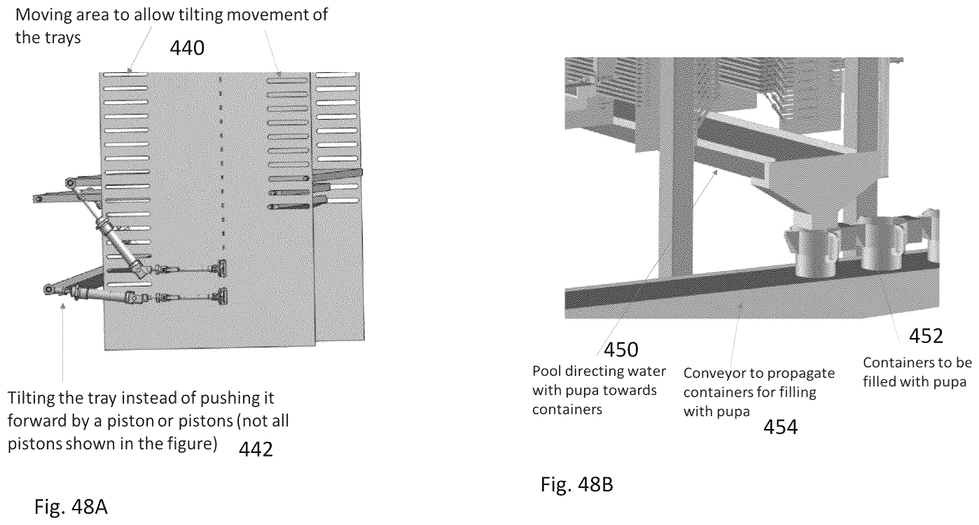

Referring now to FIG. 48A, a tray may merely be tilted instead of being pushing onto a slope. Trays are moved forward over a moving area 440 and then tilted using pistons 442. Trays may be sequentially emptied with the lowest one first and the upper most last, by contrast with the pushing method which allows separation of any tray at any given time as desired.

FIG. 48B shows a duct 450 into which the trays pour their contents. The duct then funnels the content into containers 452 on a conveyor 454. By the end of the process, there is a set of multiple containers, each with an estimated number of pupa ready for the next step in the rearing process. The pupae may be irradiated to achieve sterility, or may be placed inside cages or other means to support emergence of adult mosquitoes.

In this document, the terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. Apparatus for rearing of aquatic insects, comprising
at least one water tray, the tray having an interior tray surface and being fillable with water to provide a water surface, a water inlet leading to the water surface and a water outlet leading from the water surface, the water inlet closed with an inlet valve and the water outlet closed with an outlet valve, and mesh, the tray being arranged such that insects reared in water in the tray are above the mesh, and wherein the tray has a water cleaning configuration in which the inlet and outlet valves are open to flush clean water through the tray while the mesh holds the insects.

2. Apparatus according to claim 1, wherein the mesh is held in a frame around the tray, the frame configured to raise the mesh for said cleaning configuration, or wherein the mesh is held still and the water surface is varied.

3. (canceled)

4. Apparatus according to claim 1, wherein the tray comprises at least one rail, and a robot configured to travel a length of said tray along said rail.

5. Apparatus according to claim 4, wherein said robot comprises a first dispensing container to dispense food into said tray and optionally a second dispensing container to dispense larvae into said tray.

6. Apparatus according to claim 1, configured with an emptying mode, wherein the mesh is flush with the inner tray surface and the outlet valve is opened to empty pupae and water from the tray.

7. Apparatus according to claim 6, further comprising an image sensor and processing software to detect that a threshold level of larvae have pupated and to trigger said emptying mode.

8. Apparatus according to claim 1, comprising a rack, the rack holding a plurality of said trays, and at least one robot for processing said trays.

9. The apparatus of claim 8, wherein said robot comprises a gripper for pulling a selected one of said plurality of trays and pulling said tray to a feeding position extending in a first direction from said rack, the robot further comprising a feeding attachment for dispensing food into said tray.

10. The apparatus of claim 8, the robot comprising a piston for pushing a selected one of said trays into a pouring position to empty said tray into a collection container.

11. The apparatus of claim 10, wherein said rack comprises guide rails for guiding said tray into said pouring position.

12. The apparatus of claim 8, comprising an image sensor and image processing for determining levels of pupation in respective ones of said plurality of trays, thereby to empty respective trays only when a threshold level of pupation is met.

13. The apparatus of claim 8, wherein said robot comprises a vibrator for vibrating the robot during dispensing to provide even dispensing in said tray.

14. The apparatus of claim 1, comprising one member of the group consisting of:
an odor detector, the odor detector configured to trigger said cleaning configuration upon detection of a threshold level of a predetermined odor; and
successive trays, each with progressively larger mesh, each mesh sized for a succeeding instar, and the apparatus being configured to enter an emptying mode wherein the mesh is flush with the inner tray surface and the outlet valve is opened to empty larvae and water from the tray upon detection of a threshold number of larvae entering a next instar stage, the trays being configured that each tray empties into a next one of said successive trays.

15. (canceled)

16. Apparatus for counting and/or classification of aquatic insects, comprising:
a drainable imaging area;
a funneling mechanism for funneling water containing said aquatic insects onto said drainable imaging area; and
a sensor at said drainable imaging area for sensing said aquatic insects after said water is drained.

17. The apparatus of claim 16, wherein said drainable imaging area comprises a gated entrance for controllable entry of said aquatic insects.

18. The apparatus of claim 16, wherein said drainable imaging area comprises a movable surface to move said insects from said gated entrance to said sensor, said movable surface comprising drainage gaps for draining said water whilst retaining said insects.

19. The apparatus of claim 18, wherein said drainage gaps are mesh on said movable surface.

20. The apparatus of claim 16, wherein said funneling mechanism is a sloped surface with guide walls, or wherein said funneling mechanism is configured to receive said water and aquatic insects from a container tipped onto said funneling mechanism by a tipping mechanism.

21. (canceled)

22. The apparatus of claim 16, configured to carry out sex sorting of pupae by imaging said pupae and classifying said pupae into at least one of two classes of male and female, and placing individuals of one of said classes into a container designated for the respective class.

23. The apparatus of claim 22, wherein said classifying is based on a detected pupa size.

24. The apparatus of claim 22, wherein said classifying is based on a morphological feature.

25. The apparatus of claim 24, wherein said morphological feature is a tapering shape of an abdominal extremity of a pupa.

26. The apparatus of claim 16, configured to carry out sex sorting of larvae by engineering said larvae to fluoresce differentially depending on whether they are male or female, imaging said larvae and classifying said larvae into at least one of two classes of male and female based on fluorescence detected, and placing individuals of one of said classes into a container designated for the respective class.

27. Apparatus for rearing of aquatic insects, comprising:
a rack;
at least one water tray, the tray having an interior tray surface and being fillable with water to provide a water surface; and
a robot, the robot configured to push the tray to one side of the rack for feeding and to a second side of the rack to tip said tray to empty said aquatic insects into a collecting area.
